SEQUENCE LISTING (4 sequences)

SEQ ID NO: 1 - protein, 34 aa, synthetic construct
  MOD_RES 34: note = phenylalaninamide
  MOD_RES 1: note = pyroglutamate
XLGPQGPPHLVADPSKKQGPWLEEEEEAYGWMDX

SEQ ID NO: 2 - protein, 34 aa, synthetic construct
  MOD_RES 1: note = pyroglutamate
  MOD_RES 34: note = phenylalaninamide
XLGPQGPPHLVADPSKKQGPWLEEEEEAYGWLDX

SEQ ID NO: 3 - protein, 17 aa, synthetic construct
  MOD_RES 17: note = phenylalaninamide
  MOD_RES 1: note = pyroglutamate
XGPWLEEEEEAYGWMDX

SEQ ID NO: 4 - protein, 17 aa, synthetic construct
  MOD_RES 1: note = pyroglutamate
  MOD_RES 17: note = phenylalaninamide
XGPWLEEEEEAYGWLDX

(19) United States
(12) Patent Application Publication
(10) Pub. No.: US 2023/0407265 A1
(43) Pub. Date: Dec. 21, 2023

(54) COMPOSITION AND METHODS FOR 3D CULTURE OF ISLET CELLS

(71) Applicant: CITY OF HOPE, Duarte, CA (US)

(72) Inventors: Keiko OMORI, Duarte, CA (US); Fouad KANDEEL, Duarte, CA (US); Meirigeng QI, Duarte, CA (US)

(21) Appl. No.: 18/320,106

(22) Filed: May 18, 2023

Related U.S. Application Data
(60) Provisional application No. 63/344,490, filed on May 20, 2022.

(51) Int. Cl. C12N 5/071; A61P 3/10

(52) U.S. Cl. CPC C12N 5/0677; A61P 3/10; C12N 2501/345; C12N 2500/50; C12N 2500/34

(57) ABSTRACT

Provided herein, inter alia, are compositions and methods for culturing islet cells.

### CROSS-REFERENCE TO RELATED APPLICATIONS

This application claims the benefit of U.S. Provisional Application No. 63/344,490, filed on May 20, 2022, which is incorporated herein by reference in its entirety and for all purposes.

### REFERENCE TO A “SEQUENCE LISTING,” A TABLE, OR A COMPUTER PROGRAM LISTING APPENDIX SUBMITTED ON A COMPACT DISC

The Sequence Listing written in file 048440-803001US_SL_ST26.xml, created Sep. 8, 2023, 7,543 bytes, machine format IBM-PC, MS-Windows operating system, is hereby incorporated by reference in its entirety.

### BACKGROUND

Pancreatic islet transplantation has been proven to be a safe and effective therapy for patients with type 1 diabetes as a minimal invasive procedure. However, it's application and effectiveness are limited due to the suboptimal islet culture method to store functional islets beyond a few days.

The current method of islet culture is not optimal in maintaining islet health for long-term due to the destruction of islet microvasculature, insufficient nutrients and oxygen delivery to the core of islets, and the loss of extracellular matrix after islet isolation. Such changes in the islet microenvironment make isolated islets difficult to survive and function in vitro for more than few days. Studies show that isolated islets can survive and function better when placed in the 3D matrices such as scaffolds or hydrogels. However, there is no systematic comparative study demonstrated the survival and function of human islets before and after the culture. Such information is essential when we apply the long-term culture method for the practical therapeutic and research applications. There is no long-term islet culture method available that can maintain human islets number, viability and function longer than 2-weeks.

### BRIEF SUMMARY

In an aspect, the disclosure provides a method of culturing islet cells. The method includes: (a) obtaining islet cells from a donor and (b) incubating the islet cells in a cell culture medium including (i) a hydrogel and (ii) a gastrin compound.

In an aspect, the disclosure provides a method of treating diabetes in a subject in need. The method includes: (a) culturing islet cells according to methods provided herein, and (b) administering an effective amount of the cultured islet cells.

In an aspect, the disclosure provides an islet cell culture including islet cells, a hydrogel, a gastrin composition, and a culture medium.

In an aspect, the disclosure provides islet cells obtained by methods provided herein.

In an aspect, the disclosure provides a kit for culturing islet cells including: (i) a hydrogel and (ii) a gastrin composition.

### BRIEF DESCRIPTION OF THE DRAWINGS

FIGS. 1A-1C show islet mass recovery (FIG. 1A), islet cell purity (FIG. 1B), and islet cell viability (FIG. 1C), comparing pre-culture islet cells (Pre) to islets cultured for 4 weeks in in conventional 2D culture (4w-2D) and Vitrogel 3D hydrogel (4w-3D).

FIGS. 2A-2B show insulin release from islet cells in the glucose-stimulated insulin release assay (FIG. 2A) and the glucose stimulation index (FIG. 2B), comparing pre-culture islet cells (Pre) to islets cultured 4 weeks in conventional 2D culture versus islets cultured for 4 weeks in Vitrogel 3D hydrogel (4w-3D).

FIGS. 3A-3E show proinflammatory gene expression (ILIB (FIG. 3A), TNF (FIG. 3B), IL6 (FIG. 3C), IL8 (FIG. 3D)) and proapoptotic gene expression (BBC3 (FIG. 3E)), comparing pre-culture islet cells (Pre) to islets cultured for 4 weeks in Vitrogel 3D hydrogel (4w-3D).

FIGS. 4A-4F show R cell-associated mRNA expression of INS (FIG. 4A), MAFA (FIG. 4B), and PDX1 (FIG. 4C); a cell-associated mRNA expression of GCG (FIG. 4D), ARX (FIG. 4E); and S cell-associated mRNA expression of SST (FIG. 4F), comparing pre-culture islet cells to islets cultured for 4 weeks in Vitrogel 3D hydrogel (4w-3D).

FIGS. 5A-5E show mRNA expression of extracellular matrix genes COL4A1 (FIG. 5A), LAMA5 (FIG. 5B), CDH1 (FIG. 5C), CDH2 (FIG. 5D), GJD2 (FIG. 5E), comparing pre-culture islets with islets cultured for 4 weeks in Vitrogel 3D hydrogel (4w-3D).

FIGS. 6A-6C show immunohistochemical staining for insulin, glucagon, somatostatin, and DAPI (FIG. 6A) in pre-culture islet cells (Pre) and islets cultured for 4 weeks in Vitrogel 3D hydrogel (4w-3D). The relative area of staining for each marker in fresh islets and 4w-3D islets was quantified and compared against islets in the native pancreas (FIG. 6B). A ratio between the relative area of glucagon staining compared to insulin staining was calculated for islets in the native pancreas, pre-culture islet cells, and 4w-3D islets (FIG. 6C).

FIGS. 7A-7B show islet mass recovery (FIG. 7A), islets cultured for 4 weeks in Vitrogel 3D hydrogel without (4w-Control) and with Gastrin (4w-Gastrin); islet cell viability (FIG. 7B), comparing pre-culture islet cells to islets cultured for 4 weeks in Vitrogel 3D hydrogel without (4w-Control) and with Gastrin (4w-Gastrin).

FIGS. 8A-8B show islet cell viability (FIG. 8A), comparing islets cultured for 3 weeks in conventional 2D culture with 0 nM, 100 nM, and 600 nM gastrin added to the culture media; islet cell mass (FIG. 8B), comparing islets cultured for 3 weeks in conventional 2D culture with 0 nM, 100 nM, 300 nM, and 600 nM gastrin added to the culture media.

FIGS. 9A-9B show islet cell insulin release (FIG. 9A) and stimulation index (FIG. 9B) results from the glucose-stimulated insulin release assay, comparing pre-culture islets to islets cultured for 4 weeks in Vitrogel 3D hydrogel without (4w-Control) and with Gastrin (4w-Gastrin).

FIGS. 10A-10E show proinflammatory gene expression (ILIB (FIG. 10A), TNF (FIG. 10B), IL6 (FIG. 10C), IL8 (FIG. 10D)) and proapoptotic gene expression (BBC3 (FIG. 10E)), comparing islets cultured for 4 weeks in Vitrogel 3D hydrogel without (4w-Control) and with Gastrin (4w-Gastrin).

FIGS. 11A-11F show R cell-associated mRNA expression of INS (FIG. 11A), MAFA (FIG. 11B), and PDX1 (FIG. 11C); a cell-associated mRNA expression of GCG (FIG. 11D), ARX (FIG. 11E); and 6 cell-associated mRNA expression of SST (FIG. 4F), comparing islets cultured for 4 weeks in Vitrogel 3D hydrogel without (4w-Control) and with Gastrin (4w-Gastrin).

FIG. 12 shows gene expression for islet cell-associated and extracellular matrix genes, comparing islets cultured for 4 weeks in Vitrogel 3D hydrogel without (4w-Control) and with Gastrin (4w-Gastrin).

FIGS. 13A-13C show single cell protein expression of insulin (FIG. 13A), glucagon (FIG. 13B), and somatostatin (FIG. 13C) using Milo-scWestern blotting, comparing freshly isolated islets (Fresh) to islets cultured for 4 weeks in Vitrogel 3D hydrogel without (4w-Control) and with Gastrin (4w-Gastrin).

FIGS. 14A-14E show the blood glucose concentration in mice transplanted with islets from 3 different donors (FIG. 14A-14C), comparing freshly isolated islets to islets cultured for 4 weeks in Vitrogel 3D hydrogel (4w-3D). The arrows indicate removal of the implanted islets via nephrectomy to confirm whether the diabetes reversal was dependent on transplanted islets (FIG. 14A-C). The area under the curve (AUC) of blood glucose measurements from day 0 to 28 was quantified (FIG. 14D), comparing freshly isolated islets (Fresh) to 4w-3D islets. The percent reversal of diabetes was calculated (FIG. 14E), comparing freshly isolated islets to 4w-3D islets.

FIG. 15 shows the method of culturing human islets in the chambers for 3D culture using Vitrogel 3D hydrogels that allow islet culture growth in hydrogel while being able to provide adequate nutrients and oxygen support in the surrounding cell culture media.

FIG. 16 shows islet cell viability at week 0, 1, 2, 3, and 4, comparing islets cultured in i) our standard method, 2D-CMRL Culture Media, ii) 2D-MCDB131Culture Media, iii) 3D-VitroGel Hydrogel, and iv) 3D-GrowDex Hydrogel at 37° C.

FIGS. 17A-17E shows data comparing the effects of different hydrogel dilution and mixing ratios on long term islet survival and function. FIGS. 17A-17C show the recovery index comparing islet mass recovery (FIG. 17A), viability (FIG. 17B), and overall survival index (FIG. 17C) of islets cultured for 4 weeks in different hydrogel dilutions with pre-culture islets. FIGS. 17D-17E show islet function by comparing insulin release (FIG. 17D) and glucose stimulation index (FIG. 17E) of islet cells cultured for 4 weeks in different hydrogel dilutions with pre-culture islets.

FIGS. 18A-18C show data from an experiment optimizing 4w-3D cultured islets. FIG. 18A shows the effects of trehalose dilution of the hydrogel on islet recovery. FIGS. 18B-18C show the effects of hydrogel dilution on islet function as measured by basal insulin release (FIG. 18B) and stimulation index (FIG. 18C).

### DETAILED DESCRIPTION OF THE INVENTION

Provided herein are methods, compositions, and kits for culturing islet cells in a hydrogel with a gastrin composition. These methods and compositions allow for longer term culturing of islet cells for transplant and provide advantages of safety and convenience. Also provided are methods of treating diabetes in a subject in need

### I. Definitions

It must be noted that as used herein and in the appended claims, the singular forms “a,” “an,” and “the” include plural referents unless the context clearly dictates otherwise. It should further be understood that as used herein, the term “a” entity or “an” entity refers to one or more of that entity. For example, a nucleic acid molecule refers to one or more nucleic acid molecules. As such, the terms “a”, “an”, “one or more” and “at least one” can be used interchangeably. Similarly the terms “comprising”, “including” and “having” can be used interchangeably.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

It is appreciated that certain features of the disclosure, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about means the specified value.

As used herein, the term “gastrin” is used in accordance with its plain and ordinary meaning and refers to a peptide hormone that stimulates secretion of gastric acid (HCl) by the parietal cells of the stomach and aids in gastric motility. It is released by G cells in the pyloric antrum of the stomach, duodenum, and the pancreas. Gastrin binds to cholecystokinin B receptors to stimulate the release of histamines in enterochromaffin-like cells, and it induces the insertion of K+/H+ ATPase pumps into the apical membrane of parietal cells (which in turn increases H+ release into the stomach cavity). Its release is stimulated by peptides in the lumen of the stomach. The encoded polypeptide is preprogastrin, which is cleaved by enzymes in posttranslational modification to produce progastrin (an intermediate, inactive precursor) and then gastrin in various forms, primarily gastrin-34, gastrin-17, and gastrin-14

As used herein, the term “islet cell” or “pancreatic islet cell” refers to a cell or cells that are typically found within the Islets of Langerhans in a pancreas. Any cell normally found within the Islets of Langerhans is considered an “islet cell” or a “pancreatic islet cell”. In one embodiment, an islet cell is a “beta cell” or a “beta islet cell,” which normally produces insulin. Other cells within the islets of Langerhans include “alpha cells” or “alpha islet cells,” which normally produce glucagon, “delta cells” or “delta islet cells,” which normally produce somatostatin, “epsilon cells” or “epsilon islet cells,” which normally produce ghrelin, “gamma cells” or “gamma islet cells,” (or “PP cells) which normally produce pancreatic polypeptide (PP). Moreover, the islet cells used in the compositions and methods disclosed herein can be a mixture of one or more cell types (alpha, beta, gamma, delta and/or epsilon cells) or the islet cells used in the methods of the present invention can be a pure or substantially pure population of alpha, beta, gamma, delta and/or epsilon cells.

The cells can be from any animal, including but not limited to any mammal, such as mouse, rat, canine, feline, bovine, equine, porcine, non-human and human primates. Mammalian cells particularly suitable for cultivation in the present media include islet cells of human origin, which may be primary cells derived from a pancreas. In addition, transformed cells or established cell lines islet cell lines can also be used. The cells used herein can be normal, healthy cells. The cells can be from donor with a healthy pancreas. In embodiments, the cells are not primary cells, such as cells from an established cell line, transformed cells, thawed cells from a previously frozen collection and the like. Animal cells for culturing by the present invention may be obtained commercially, for example from ATCC (Rockville, Md.), Cell Systems, Inc. (Kirkland, Wash.), Clonetics Corporation (San Diego, Calif.), BioWhittaker (Walkersville, Md.) or Cascade Biologicals (Portland, Oreg.).

By “cell culture” or “culture” is meant the maintenance of the cells in an artificial, in vitro environment. The term “cell culture” also encompasses cultivating individual cells and tissues. The cells being cultured according to the present invention, whether primary or not, can be cultured and plated or suspended according to the disclosed conditions. The examples herein demonstrate at least one functional set of culture conditions that can be used in conjunction with the methods described herein. If not known, plating or suspension and culture conditions for a given animal cell type can be determined by one of ordinary skill in the art using only routine experimentation. Cells may or may not be plated onto the surface of culture vessels, and, if plated, attachment factors can be used to plate the cells onto the surface of culture vessels. If attachment factors are used, the culture vessels can be precoated with a natural, recombinant or synthetic attachment factor or factors or peptide fragments thereof, such as but not limited to collagen, fibronectin and natural or synthetic fragments thereof.

As used herein, the term “culturing islet cells” is used in accordance with its plain and ordinary meaning and refers to the process by which cells are grown under controlled conditions, generally outside their natural environment. After the cells of interest, herein islets cells, have been isolated from living tissue, they can subsequently be maintained under carefully controlled conditions. These conditions vary for each cell type, but generally consist of a suitable vessel with a substrate or medium that supplies the essential nutrients.

As used herein, the term “obtaining islet cells” is used in accordance with its plain and ordinary meaning and refers to the process by which interested cells, herein islets cells, can be isolated from solid tissues by digesting an extracellular matrix using enzymes such as collagenase, trypsin, or pronase, before agitating the tissue to release the cells into suspension. Islet cells for use in the present invention can be obtained, for example from human donor pancreases. In islet cell transplantation procedures, surgeons use enzymes to obtain islet cells, typically from the pancreata of multiple deceased donors, in order to collect an ample amount of cells that can be immediately injected into the recipient's liver.

As used herein, the term “donor” is used in accordance with its plain and ordinary meaning and refers to an individual organism that supplies living tissue to be used in another body, as a person who furnished blood for transfusion or an organ for transplantation in a histo-compatible recipient. In embodiments, the donor is a living human donor. In embodiments, the donor is a deceased human donor. In embodiments, the donor is a living human donor who does not have pre-diabetes, Type 1 diabetes, or Type 2 diabetes. In embodiments, the donor is a deceased human donor who did not have pre-diabetes, Type 1 diabetes, or Type 2 diabetes.

As used herein, the term “incubating” is used in accordance with its plain and ordinary meaning and refers to a process of contacting one or more components of a reaction with another component or components, under conditions and for sufficient time such that a desired reaction product is formed. The term “incubating” is used to describe a particular step in which a cell or group of cells is regulated. The incubating process may include regulating a particular temperature, reagent, or condition of the cell or group of cells.

As used herein, the term “cell culture medium” is used in accordance with its plain and ordinary meaning and encompasses any gel or liquid created to support cellular growth in an artificial environment. A culture medium plays an integral role in cell culture technology, supporting in vitro cellular research. It is the medium that supplies the nutrients necessary for cell cultures to survive and proliferate. The cell culture medium also provides the correct osmolality and pH. There are a variety of different types of cell culture media that accommodate cells from mammals, plants, insects, bacteria, yeast, viruses, and more. The term cell culture medium may be used interchangeably with cell medium or culture medium. In embodiments, cell culture medium may include, but not limited to, any of the hydrogels and a gastrin compound.

As used herein, the term “hydrogel” is used in accordance with its plain and ordinary meaning and refers to a network of crosslinked polymer chains that are hydrophilic in nature. Further, hydrogel may be found as a colloidal gel in which water is a dispersion medium. Hydrogels are highly absorbent (they can contain over 90% water) natural or synthetic polymeric networks. The hydrophilic polymer chains being held together by cross-links may result into a three-dimensional solid. The crosslinks which bond the polymers of the hydrogel may fall under categories such as physical and chemical. Because of the inherent cross-links, the structural integrity of the hydrogel network does not dissolve from the high concentration of water.

As used herein, the term “diabetes” is used in accordance with its plain and ordinary meaning and refers to a group of metabolic disorders characterized by a high blood sugar level over a prolonged period of time. Malfunctioning of pancreas as not producing enough insulin may lead to diabetes. Further, improper functioning of body cells towards the produced insulin may also cause diabetes.

As used herein, the term “subject in need” is used in accordance with its plain and ordinary meaning and refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In embodiments, a subject is human.

As used herein, the term “effective amount” is used in accordance with its plain and ordinary meaning and refers to an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “administering” is used in accordance with its plain and ordinary meaning and refers to a means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

As used herein, the term “post administration” is used in accordance with its plain and ordinary meaning and refers to the interval following administration of a drug.

As used herein, the term “pre administration” is used in accordance with its plain and ordinary meaning and refers to the interval before administration of a drug.

As used herein, the term “dosage” is used in accordance with its plain and ordinary meaning and refers to the rate of application of a dose. Moreover, the dose refers to quantity (in units of energy/mass) in the fields of nutrition, medicine, and toxicology. Dosages may vary depending upon the requirements of the subject in need and the compound being employed. The dose administered to a subject in need, in the context of the present disclosure, should be sufficient to effect a beneficial therapeutic response in the subject in need over time. The size of the dose also will be determined by the existence, nature, and extent of any adverse side-effects. Determination of the proper dosage for a particular situation is within the skill of the practitioner. Dosage amounts and intervals can be adjusted individually to provide levels of the administered compound effective for the particular clinical indication being treated. This will provide a therapeutic regimen that is commensurate with the severity of the individual's disease state.

As used herein, the term “proton pump inhibitor” is used in accordance with its plain and ordinary meaning and refers to potent inhibitors of acid secretion. Moreover, proton-pump inhibitors function as profound and prolonged reduction of stomach acid production. Proton pump inhibitors act by irreversibly blocking the hydrogen/potassium adenosine triphosphatase enzyme system such as the H+/K+ ATPase, or, more commonly, the gastric proton pump of gastric parietal cells. The proton pump is the terminal stage in gastric acid secretion, being directly responsible for secreting H+ ions into the gastric lumen, making it an ideal target for inhibiting acid secretion. Proton pump inhibitors include omeprazole, esomeprazole, lansoprazole, dexlansoprazole, pantoprazole, rabeprazole, and ilaprazole.

As used herein, the term “DPP-4 inhibitor” is used in accordance with its plain and ordinary meaning and refers to a class of oral hypoglycemics that block the enzyme dipeptidyl peptidase-4 (DPP-4). They can be used to treat diabetes mellitus type 2. DPP-4 inhibitors reduce glucagon and blood glucose levels. The mechanism of DPP-4 inhibitors is to increase incretin levels (GLP-1 and GIP), which inhibit glucagon release, which in turn increases insulin secretion, decreases gastric emptying, and decreases blood glucose levels. DPP-4 inhibitors include sitagliptin, vildagliptin, saxagliptin, linagliptin, gemigliptin, anagliptin, teneligliptin, alogliptin, trelagliptin, omarigliptin, evogliptin, gosogliptin, and dutogliptin.

As used herein, the term “Type 1 diabetes” is used in accordance with its plain and ordinary meaning and refers an autoimmune disease that is a form of diabetes in which very little or no insulin is produced by islets of Langerhans (containing insulin-producing beta cells) in the pancreas. The underlying mechanism involves an autoimmune destruction of the insulin-producing beta cells in the pancreas. This results in high blood sugar levels in the body. Symptoms of Type 1 diabetes (T1D) may include frequent urination, increased thirst, increased hunger, and weight loss. Additionally, or alternatively, the symptoms may include blurry vision, tiredness, and slow wound healing. The symptoms typically develop over a short period of time.

As used herein, the term “Type 2 diabetes” is used in accordance with its plain and ordinary meaning and refers a disease characterized by high blood sugar, insulin resistance, relative lack of insulin and so on. Type 2 diabetes is herein interchangeably referred as the adult-onset diabetes. Symptoms of Type 2 diabetes may include increased thirst, frequent urination, unexplained weight loss, hunger, feeling tired, and sores that do not heal. Additionally, or alternatively, long-term complications from high blood sugar may include heart disease, strokes, and diabetic retinopathy.

As used herein, the term “insulin-independent” is used in accordance with its plain and ordinary meaning and refers to diabetic subject who becomes insulin free (that is, requiring no injectable insulin).

As used herein, the term “kidney transplant” is used in accordance with its plain and ordinary meaning and refers to an organ transplant of a kidney into a subject in need. A kidney transplant is a surgical procedure to place a healthy kidney from a living or deceased donor into a person whose kidneys no longer function properly. A kidney transplant may be an option if the kidneys do not function adequately. When kidney function is so poor that is it life-threatening, it is called end-stage renal disease (ESRD) or end-stage kidney disease (ESKD). A kidney transplant may be classified as a deceased-donor/cadaveric or living-donor transplantation depending on the source of a donor organ. Further, living-donor kidney transplants may be characterized as genetically related (living-related) or non-related (living-unrelated) transplants, depending on whether a biological relationship exists between a donor and a recipient.

### II. Compositions

In an aspect, provided herein are compositions comprising cultures of islet cells, wherein donor islet cells are suspended in a hydrogel and a gastrin compound.

The term “gastrin compounds” as used herein means agents that bind to, interacts with or stimulates the gastrin/CCK receptor. Gastrin compounds include gastrin derivatives and conjugates as well as peptide homologs, that are capable of interacting with the gastrin/CCK receptor. The terms “derivatives” and “conjugates” as used herein are equivalent, and are used to indicate compositions that are chemically related, and can be prepared by synthetic, biological, recombinant or chemical means.

The modified gastrin can be a gastrin derivative or analog comprising a minimal sequence of 6 amino acids (from the C-terminal end), and further having addition of a reactive group such as a cysteine residue capable of undergoing an addition reaction (Refer to SEQ ID NO:1-4). In embodiments, the gastrin may extend up to 34 amino acids (“Big” Gastrin or Gastrin-34), wherein at least one reactive amino acid such as a cysteine residue or a lysine residue is added or substituted at the N-terminal end. The addition of the reactive amino acid such as a cysteine can be at a terminal region, and in related embodiments, a spacer can optionally precede the added reactive amino acid. For example, the spacer can be synthesized biologically as part of, or can be chemically attached to the gastrin amino acid sequence, forming a structure which has a gastrin sequence-spacer-cysteine. For instance, the spacer can be a sequence of several amino acids such as alanine or glycine. The sequence of amino acids can be alternating amino acids (e.g. glycine/alanine) or can be non-alternating, i.e., can be a random sequence or a particular sequence. The sequence can consist of at least one amino acid.

In embodiments, a bifunctional cross-linking agent which is a reactive component is added to the modified gastrin, particularly to the gastrin having an added reactive group at the amino terminus (e.g., a cysteine), or to a modified gastrin having a spacer, via a homobifunctional or heterobifunctional portion of the crosslinker to generate an a modified gastrin having a reactive group such as a thiol of an amino group at one end (e.g., to form, as listed from the carboxy terminus, a gastrin-spacer-cys-cross-linker-carrier; a gastrin-cys-cross-linker group-carrier; gastrin-spacer-cys-cross-linker with reactive group exposed, and a gastrin-cys-cross-linker with reactive group exposed.)

The modified gastrin can be further conjugated in vitro to one or more plasma components such as whole or fractionated serum obtained from the subject in need; one or more purified serum protein(s) such as albumin, transferrin or an immunoglobulin; lipids/lipophilic moieties/hydrophobic moieties; or to polymeric carriers such as dextran or PEG prior to injection. The term polymer as used herein includes polymers of amino acids, sugars, nucleosides, synthetic polymers (such as PEG) and mixtures thereof. The polymer can be activated, for instance with a bifunctional crosslinker or via other chemical means prior to conjugation.

The gastrin compositions can be associated with a larger molecule such as a polymer, either non-covalently, or as a covalent conjugate, or as a fusion protein to another peptidic compound having an amino acid sequence. The gastrin compounds can have a longer half-life in circulation in a subject in need, and/or maintain higher concentrations in vivo of the gastrin compounds for an extended period of time compared to the native forms of gastrin.

The compositions can further include at least one growth factor, a hypoglycemic agent, or an immunosuppressant, for the treatment of diabetes. Examples of growth factors include but are not limited to a EGF receptor ligand such as EGF, a GLP-1 receptor ligand such as GLP-1, prolactin receptor ligand such as prolactin and growth hormone receptor ligand such as growth hormone. Examples of immunosuppressants include but are not limited to cyclosporine, FK506, rapamycin, and daclizumab. Non-limiting examples of hypoglycemic agents include sulfonylureas, meglitinides, biguanides, thiazolidinediones, and alpha-glucosidase inhibitors.

In embodiments, a gastrin compound can be bound to a comparatively larger structure or a plurality of structures in the blood and still retain the ability to bind target proteins, i.e., a gastrin/CCK receptor. Generally, gastrin, which would be otherwise rapidly degraded in the body, is attached to a carrier protein; using this composition, a longer-term of drug efficacy can be achieved. Alternatively a gastrin compound can be conjugated to a polymeric carrier such as a polyethylene glycol (PEG) or a dextran to achieve similar objectives

In embodiments, chemical modification of gastrin is used to provide compounds that react covalently or non-covalently to carrier proteins or polymeric carriers, either in vitro (ex vivo) or in vivo. In embodiments, the non-covalent interaction is electrostatic or hydrophobic. In embodiments, the carrier protein is a plasma protein. In embodiments, the plasma protein is an albumin or an immunoglobulin or components of an immunoglobulin. The immunoglobulin or components of the immunoglobulin can be modified or portions deleted prior to conjugation. In embodiments, the polymeric carrier is polyethylene glycol or dextran. For instance, activated PEG can be attached to gastrin compound via an amino group in the gastrin compound (Vernonese, F M. Biomaterials 22 (2001)-405-41.7).

In embodiments, the gastrin compound which is a sequence of amino acids, is genetically fused with the carrier protein, which is also a sequence of amino acids using standard recombinant genetic techniques. Gastrin can be fused recombinantly to a carrier protein with or without a linker/spacer, for example, comprising a sequence of small neutral uncharged amino acids. A nucleic acid encoding gastrin can be recombinantly fused or synthesized directly as a fusion to portions or the whole of the carrier protein and the nucleic acid construct or fusion protein can encode or incorporate a number of additional amino acids to act as a spacer between the two proteins. Recombinant fusion proteins can be expressed in yeast (Saccharomyces, Pichia) or in standard bacterial systems, or mammalian or insect cell systems can be used. Following standard procedures for expression and/or purification, the fusion protein can be used therapeutically. Modifications to the sequence of gastrin compound polypeptide can be introduced during construction of the fusion protein if necessary.

In embodiments, the gastrin compound is modified to introduce a reactive group such as those present on an amino acid such as a lysine or cysteine so that the reactive group upon further contacting another compound such as a carrier protein or carrier non-proteinaceous polymer, can form covalent interactions with the carrier proteins or polymers. For instance, a reactive thiol group can be added to the gastrin molecule through an amino group on lysine, for example, using succinimidyl 3-2-pyridyldithio propionate (SPDP) followed by reduction with DTT to release the active thiol group ((“Protein thiolation and reversible protein-protein conjugation. N-Succinimidyl 3-(2-pyridyldithio)propionate, a new heterobifunctional reagent.” Carlsson J, Drevin H, Axen R. Biochem J 173, 723-737 (1978)). Further, the bifunctional group can also be added after the cysteine or lysine has been added, so that one reactive end of the crosslinking agent will react with cysteine/lysine while the other reactive end at the other end is left exposed or is conjugated to a carrier.

Thiols can be also incorporated at carboxylic acid groups by EDAC-mediated reaction with cystamine, followed by reduction of the disulfide with DTT. (“Introduction of sulfhydryl groups into proteins at carboxyl sites.” Lin C M, Mihal K A, Krueger R J. Biochim Biophys Acta 1038, 382-385 (1990). In a non-limiting example, reaction of an amino group on the lysine residue in gastrin with succinimidyl trans-4-(maleimidylmethyl)cyclohexane-1-carboxylate (“Conjugation of glucose oxidase from Aspergillus niger and rabbit antibodies using N-hydroxysuccinimide ester of N-(4-carboxycyclohexylmethyl)-maleimide.” Yoshitake S, Yamada Y, Ishikawa E, Masseyeff R. Eur J Biochem 101, 395-399 (1979)) introduces a thiol reactive group at amino sites of gastrin that can subsequently react with cysteine residues of the carrier protein or free thiol group on the activated polymer.

A gastrin compound-carrier complex can include additional modular components including a spacer or element or other component that can facilitate preparation or isolation of the gastrin compound-carrier complex or enhance or maintain the functional activity of the gastrin compound. The spacer can be one or more amino acids, peptide, a peptidomimetic, or a small organic molecule, and can comprise homobifunctional or heterobifunctional crosslinking agents or chitin oligomers or polyethylene glycol or related polymers.

In embodiments, the carrier and gastrin compound are covalently crosslinked with or without a spacer. Examples of non-spacers (zero-length crosslinkers) include EDC. Homobifunctional crosslinkers that generate a spacer can be for instance disuccinimidyl suberate and heterobifunctional crosslinkers that generate a spacer can be for instance 2-iminothiolane, succinimidyl 6-[3-(2-pyridyldithio)propionamido] hexanoate (LC-SPDP) and 4-(N-maleimido methyl)cyclohexane-1-carboxylate (SMCC).

In embodiments, the gastrin compound is associated with a larger carrier moiety such as a polymer, for example a protein. As the association may be covalent or non-covalent, the protein may be considered to be a carrier protein. Classes of carrier proteins can possess the properties of being non-antigenic, i.e., are native human proteins, and are being capable of sustained maintenance in circulation. An ideal carrier protein is one normally found in the human circulatory system.

As used herein, the term “gastrin/CCK receptor ligand” encompasses any compound, that binds to, interacts with or stimulates the gastrin/CCK receptor. Examples of such gastrin/CCK receptor ligands are given in U.S. Pat. No. 6,288,301, and include various forms of gastrin, such as gastrin 34 (big gastrin), gastrin 17 (little gastrin or small gastrin), gastrin 14, gastrin 13, gastrin-10, and gastrin 8, pentagastrin, tetragastrin; various forms of cholecystokinin such as CCK 58, CCK 33, CCK 22, CCK 12 and CCK 8; and other gastrin/CCK receptor ligands. In general, gastrin/CCK receptor ligands share a carboxy terminal amino acid sequence Trp-Met-Asp-Phe-amide. The aforementioned methionine (Met) can be replaced by a leucine. Also contemplated are active analogs, fragments and other modifications of the above, including both peptide and non-peptide agonists or partial agonists of the gastrin/CCK receptor such as A71378 (Lin et al., Am. J. Physiol. 258 (4 Pt 1): G648, 1990).

Small forms of gastrin such as gastrin 17 are economically prepared by peptide synthesis, and synthetic peptides are commercially available. Synthetic human gastrin 17 such as human gastrin 17 having methionine or leucine at position 15 are also available from Bachem A G, Bubendorf, Switzerland, and from Researchplus. Gastrin peptides as found in nature are carboxyl-terminally amidated peptides, and amidation of the carboxyl terminus amino acid is within the scope of gastrin compounds herein.

Gastrin/CCK receptor ligands include also active analogs, fragments and other modifications of the above ligands, which for example share amino acid sequence with an endogenous mammalian gastrin, for example, share 60% sequence identity, or 70% identity, or 80% identity. Such ligands also include compounds that increase the secretion of endogenous gastrins, cholecystokinins or similarly active peptides from sites of tissue storage. Examples of these are the gastric releasing peptide, omeprazole which inhibits gastric acid secretion, and soya bean trypsin inhibitor which increases CCK stimulation

The sequence of big gastrin-34 and small gastrin-17 are shown herein. Big gastrin-34 is essentially an extension form of small gastrin-17 having an additional amino acid sequence at the N-terminal end. Big gastrin is cleaved in vivo to release gastrin-17. The symbol “Glp” at the N-terminal end is a pyroglutamate residue, which is a naturally cyclized form of glutamate. In various embodiments, gastrins having an N-terminal pyroglutamate residues are modified to contain N-terminal cysteine or lysine residues by either replacing the pyroglutamate with a glutamate or glutamine, or deleting the pyroglutamate. Further, each of a gastrin 34 and gastrin-17 can be used in a modified form that has a methionine or a leucine at position 32 as shown herein in SEQ ID No: 1-2, respectively, or at position 15 as shown in SEQ ID No: 3-4, respectively. The symbol “Phe-NH2” is a phenylalaninamide residue.

N-terminal

(SEQ ID NO: 1)

Glp-Leu-Gly-Pro-Gln-Gly-Pro-Pro-His-Leu-Val-Ala-
Asp-Pro-Ser-Lys-Lys-Gln-Gly-Pro-Trp-Leu-Glu-Glu-
Glu-Glu-Glu-Ala-Tyr-Gly-Trp-Met-Asp-Phe-NH2.
N-terminal

(SEQ ID NO: 2)

Glp-Leu-Gly-Pro-Gln-Gly-Pro-Pro-His-Leu-Val-Ala-
Asp-Pro-Ser-Lys-Lys-Gln-Gly-Pro-Trp-Leu-Glu-Glu-
Glu-Glu-Glu-Ala-Tyr-Gly-Trp-Leu-Asp-Phe-NH2.
N-terminal

(SEQ ID NO: 3)

Glp-Gly-Pro-Trp-Leu-Glu-Glu-Glu-Glu-Glu-Ala-Tyr-
Gly-Trp-Met-Asp-Phe-NH2.
N-terminal

(SEQ ID NO: 4)

Glp-Gly-Pro-Trp-Leu-Glu-Glu-Glu-Glu-Glu-Ala-Tyr-
Gly-Trp-Leu-Asp-Phe-NH2.

In embodiments, the hydrogel comprises Gellan gum. Gellan gum is a water-soluble anionic capsular polysaccharide produced by the bacterium Sphingomonas elodea (formerly Pseudomonas elodea). Hydrogels comprising Gellan gum are described in U.S. Pat. No. 10,603,406. Commercial versions include Vitrogel® 3D and Vitrogel® MMP manufactured by TheWell Bioscience (North Brunswick, NJ).

Gellan gum is manufactured by fermenting an appropriate strain of Sphingomonas with a readily available carbohydrate source. The constituent sugars of gellan gum are glucose, glucuronic acid and rhamnose in the molar ratio of about 2:1:1. These are linked together to give a primary structure comprising a linear tetrasaccharide repeat unit (O'Neill M. A. et al., Carbohydrate Research, Vol. 124, p. 123, 1983; Jansson, P. E. et al., Carbohydrate Research, Vol. 124, p. 135, 1983). In the native or high acyl form of gellan gum, two acyl substituents, acetate and glycerate, are present. Both substituents are located on the same glucose residue and, on average, there is one glycerate per repeat unit and one acetate per every two repeat units. In the low acyl form of gellan gum, the acyl groups have been removed to produce a linear repeat unit substantially lacking such groups. Deacylation of the gum is usually carried out by treating a fermentation broth with alkali.

The high acyl form of gellan gum does not require addition of any substances for gel formation provided the gum concentration is higher than the critical concentration. High acyl gellan gum produces a soft, elastic, and non-brittle gel when its solution is cooled below the setting temperature. High acyl gellan gum gels will soften with heat and melt at a temperature proximate to the setting temperature. Low acyl gellan gum polymers typically have a range of the degree of acylation from about 1 to 2 glycerate per repeat and 1 to 2 acetate per every two repeats. The low acyl form of gellan gum generally requires a gelation agent such as salt or acid for gel formation. For example, low acyl gellan gum forms a firm, non-elastic, and brittle gel when cooled in the presence of gel-promoting cations, preferably divalent cations, such as calcium and magnesium.

In general, gellan gum as described above can dissolve in water at the temperature higher than 0° C. at a concentration of 0.001% to 10% w/v, while gellan gum of all types can dissolve completely in water at a temperature higher than 80° C. The gellan gum aqueous solution thus formed can maintain in a liquid form after dissolution or heating-cooling circle at temperature higher than 0° C. and pH of about 4-10.

Two types of hydrogel with different rheological properties can be formed depending on the mixing ratios and ionic concentrations of solutions forming the hydrogel. A soft hydrogel comprising a fiber structure can be formed when the gellan gum solution and trigger solution are mixed from 100:1 to 1:1 ratios. Preferably, the mixing ratio is 4:1 to 1:1. The soft hydrogel possesses a shear thinning and self-healing rheological property, which allow the hydrogel be converted into a liquid state by shearing force (such as pipetting, syringe injection or pump perfusion) but rapidly recover its hydrogel state once the external force is ceased. The gel-sol state can be transformed multiple times. Cells and biomolecules can be embedded within the hydrogel and deliver to a different location by injection. The mixing is typically performed at a temperature from about 4 to about 60° C., preferably at room temperature to about 37° C. Ion trigger solution contains one or more positive ionic molecular such as Na+, K+, Ca++, Mg++ etc. The ionic concentration higher than 0.01%.

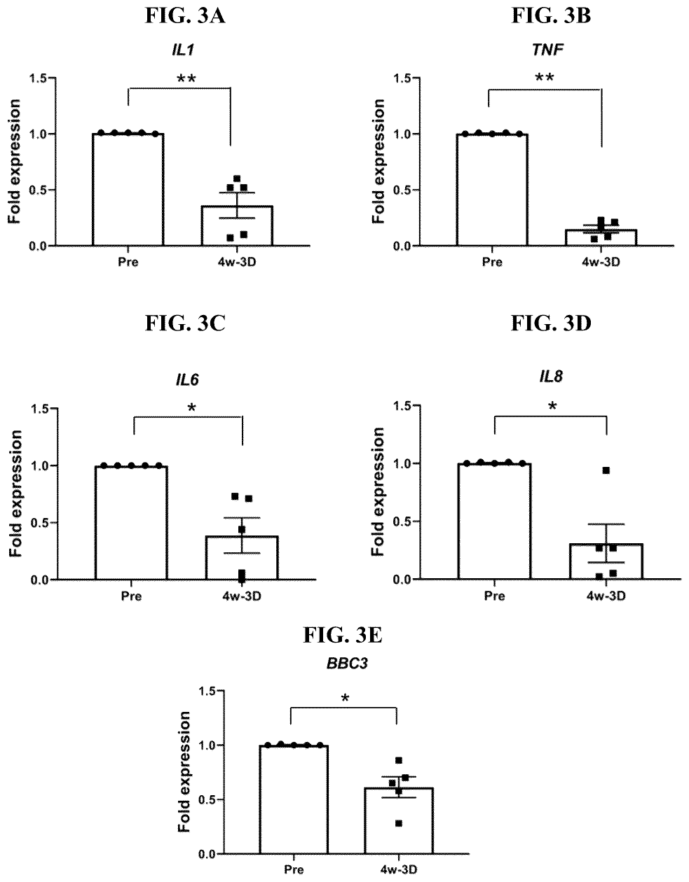

A hard hydrogel comprising an agglomeration structure can be formed with the gellan gum solution and trigger solution are mixed from 1:1 to 1:100 ratios or when the trigger solution contain high ionic concentration. Preferably, as shown in FIG. 3, the mixing ratio is 1:1 to 1:4 and the trigger solution has an ion (e.g., Ca2+) concentration higher than 0.02% w/v. In a preferred embodiment, the mixing range for hydrogel formation is 4:1 v/v (4 parts of gellan gum solution mixed with 1 part of cell culture medium) to 1:4 (1 part of gellan gum solution mixed with 4 part of cell culture medium). The hard hydrogel is stiff and brittle and doesn't possess the shear thinning and self-healing rheological property. When disturbed with external force, the hard hydrogel can be broke into small gel particles. The hard hydrogel can maintain its hydrogel formation when it is placed in an 80° C. water bath. In the aforementioned preferred embodiment, the hard hydrogel formed can maintain its hydrogel formation at a temperature as high as 80° C.

Additionally, the soft gel can be converted to hard gel when an additional ionic solution is added into the hydrogel system, such as by covering with or submerging in extra phosphate buffer, cell culture media or ionic solutions. As an example: mixing 800 μL 1% gellan gum solution with 200 μL DMEM medium will form a soft gel. After soft gel is formed, adding 1 mL DMEM medium on the top of the soft gel will convert the soft hydrogel into hard hydrogel within 12 hours.

Provided herein are islet cells obtained by any of the methods for culturing islet cells described herein. Proinflammatory cytokine genes (ILIB, TNF, and IL6) and proapoptotic gene (BBC3) gene expression is significantly decreased and islet function is maintained in vitro in islet cells so obtained, in comparison to pre-culture islet cells.

### III. Kits

In an aspect provided herein are kits for culturing islet cells comprising a hydrogel and a gastrin compound. The kits can include any of the hydrogels or gastrin compounds disclosed above. The kits can also include culture media. The kits can also include instructions for use.

### IV. Methods

Provided herein are methods for culturing donor islet cells in a 3D hydrogel with a gastrin compound. The applicants found that, surprisingly, the islet cells in 3D culture with a gastrin compound could be maintained in culture for much longer than donor islet cells in a standard 2D culture. As a consequence, the medical professional has much greater flexibility is timing an islet cell transplant to a subject in need Moreover, for the subject that also requires a kidney transplant, provides convenience in coordinating the islet cell transplant with the kidney transplant to the same subject in need. Consequently, the medical procedures can be performed with increased safety to the subject in need.

In subjects with diabetes and end stage renal disease, kidney transplantation together with same donor islet transplant therapy has many advantages over kidney transplant alone including: a) improving kidney graft survival, b) reducing risks for cardiovascular disease and other diabetes complications, and c) improving overall patient survival. It will also save the patients the very high risk associated with solid pancreas transplantation, as solid pancreas transplant surgery is very extensive and carry significant morbidities. Currently in the United States, less than 1000 simultaneous pancreas and kidney transplantations are performed yearly in both type 1 and type 2 diabetics, due to either poor quality of available donated pancreata or poor health of potential recipients who may not sustain the high surgical risk and complications of pancreas transplant surgery. However, pancreas suitability for islet transplantation is much less stringent than the pristine quality frequently required for solid organ transplant, allowing for the utilization of much larger number of available donors pancreata. Further, single donor kidney and islets that can be done consecutively over a short period of time would have significant advantages over unrelated kidney islet transplants that are done over a longer span of time due to: a) subjecting the receiving patient to a single load of allo antigen, which reduces the immunological burden and associated higher risk for renal graft rejection; and b) eliminating the need for repeating immune suppression induction that requires longer hospital stay, higher cost and increased morbidity. In addition, performing islet transplantation from the same donor within a few weeks after same donor kidney transplantation will not require the recipient patient to remain under anesthesia for extended period of time required for islet isolation when kidney and islet are transplanted in the same surgical setting, save transplanted islets the initial impact of the immune response to allo antigen exposure caused by the kidney transplant, and provides the islets with the added protection offered by the engrafted kidney with the same allo antigen signature.

In an aspect, the methods for culturing donor islet cells include obtaining islet cells and incubating the cells in a cell culture medium comprising (i) any of the hydrogels disclosed above and (ii) a gastrin compound. The islet cells can be obtained from a healthy donor. The methods can include any of the hydrogel composition or gastrin compositions disclosed above. The incubating can be for more than 3 days, more than 4 days, more than 5 days, or more than 6 days. In embodiments, the incubating is for at least a week, at least 2 weeks, at least 3 weeks, at least 4 weeks, at least 5 weeks, at least 6 weeks, at least 7 weeks, or at least 8 weeks. In embodiments, the incubating is from about 3 days to about 6 weeks. In embodiments, the incubating is from about 3 days to about 4 weeks. In embodiments, the incubating is from about 3 days to about 2 weeks. In embodiments, the incubating is from about 3 days to about 1 week.

In embodiments the methods of culturing islet cells includes a gastrin compound, wherein the concentration of gastrin is about 100 nM to about 600 nM. In embodiments, the concentration of gastrin is preferably from about 250 nM to about 300 nM. In embodiments, the concentration of gastrin is about 100 nM to about 150 nM. In embodiments, the concentration of gastrin is about 100 nM to about 200 nM. In embodiments, the concentration of gastrin is about 100 nM to about 250 nM. In embodiments, the concentration of gastrin is about 100 nM to about 300 nM. In embodiments, the concentration of gastrin is about 100 nM to about 350 nM. In embodiments, the concentration of gastrin is about 100 nM to about 400 nM. In embodiments, the concentration of gastrin is about 100 nM to about 450 nM. In embodiments, the concentration of gastrin is about 100 nM to about 500 nM. In embodiments, the concentration of gastrin is about 100 nM to about 550 nM. In embodiments, the concentration of gastrin is about 150 nM to about 600 nM. In embodiments, the concentration of gastrin is about 200 nM to about 600 nM. In embodiments, the concentration of gastrin is about 250 nM to about 600 nM. In embodiments, the concentration of gastrin is about 300 nM to about 600 nM. In embodiments, the concentration of gastrin is about 350 nM to about 600 nM. In embodiments, the concentration of gastrin is about 400 nM to about 600 nM. In embodiments, the concentration of gastrin is about 450 nM to 600 nM. In embodiments, the concentration of gastrin is about 500 nM to about 600 nM. In embodiments, the concentration of gastrin is about 550 nM to about 600 nM. In embodiments, the concentration of gastrin is about 150 nM to about 550 nM. In embodiments, the concentration of gastrin is about 200 nM to about 500 nM. In embodiments, the concentration of gastrin is about 200 nM to about 400 nM. In embodiments, the concentration of gastrin is about 225 nM to about 325 nM.

In embodiments the methods of culturing islet cells further includes trehalose, wherein the concentration of trehalose is about 0.01 M to about 0.35 M. In embodiments, the concentration of trehalose is about 0.01 M to about 0.05 M. In embodiments, the concentration of trehalose is about 0.01 M to about 0.1 M. In embodiments, the concentration of trehalose is about 0.01 M to about 0.15 M. In embodiments, the concentration of trehalose is about 0.01 M to about 0.2 M. In embodiments, the concentration of trehalose is about 0.01 M to about 0.25 M. In embodiments, the concentration of trehalose is about 0.01 M to about 0.3 M. In embodiments, the concentration of trehalose is about 0.01 M to about 0.35 M. In embodiments, the concentration of trehalose is about 0.05 M to about 0.35 M. In embodiments, the concentration of trehalose is about 0.1 M to about 0.35 M. In embodiments, the concentration of trehalose is about 0.15 M to about 0.35 M. In embodiments, the concentration of trehalose is about 0.2 M to about 0.35 M. In embodiments, the concentration of trehalose is about 0.25 M to about 0.35 M. In embodiments, the concentration of trehalose is about 0.3 M to about 0.35 M. In embodiments, the concentration of trehalose is about 0.1 M to about 0.3 M. In embodiments, the concentration of trehalose is about 0.15 M to about 0.25 M. In embodiments, the concentration of trehalose is about 0.18 M to about 0.22 M. In embodiments, the concentration of trehalose is about 0.19 M to about 0.21 M. In embodiments, the concentration of trehalose is about 0.2 M.

Also provided herein are methods for treating diabetes in a subject in need by culturing islet cells according to any of the methods disclosed above and administering the cultured islet cells to the subject in an effective amount. The method of treating diabetes can further include administering a gastrin compound to the subject. Administering a gastrin compound can be pre-administration of the cultured islet cells, post-administration of the cultured islet cells, or both. The gastrin compound can be administered to the subject daily, twice daily, or more. The gastrin compound can be administered for at least 5, at least 10, at least 15, at least 20, at least 25, at least 30, at least 40, at least 50, at least 60, or more days. In embodiments, the gastrin compound is administered daily for about 1 day to about 60 days. In embodiments, the gastrin compound is administered daily for about 14 day to about 42 days. In embodiments, the gastrin compound is administered daily for about 30 days. In embodiments, the gastrin compound is administered twice daily for about 1 day to about 60 days. In embodiments, the gastrin compound is administered twice daily for about 14 day to about 42 days. In embodiments, the gastrin compound is administered twice daily for about 30 days. In embodiments, the gastrin compound is gastrin-34, gastrin-17, or gastrin-14. In embodiments, the gastrin compound is gastrin-34. In embodiments, the gastrin compound is gastrin-17. In embodiments, the gastrin compound is gastrin-14. In embodiments, the gastrin compound is administered in an amount from about 0.001 microgram to about 10 grams. In embodiments, the gastrin compound is administered in an amount from about 0.001 microgram to about 1 gram. In embodiments, the gastrin compound is administered in an amount from about 0.01 microgram to about 0.1 gram. In embodiments, the gastrin compound is administered in an amount from about 0.01 microgram to about 0.01 gram. In embodiments, the gastrin compound is administered in an amount from about 0.01 microgram to about 1,000 micrograms. In embodiments, the gastrin compound is administered in an amount from about 0.01 microgram to about 100 micrograms. In embodiments, the gastrin compound is administered in an amount from about 0.01 microgram to about 10 micrograms. In embodiments, the gastrin compound is administered in an amount from about 0.01 microgram to about 1 microgram.

Skilled medical professionals can adjust dosage amounts and schedules as appropriate so as for the subject to attain insulin independence. Treatment of the subject can also include administering an effective amount of a proton pump inhibitor. Examples of proton pump inhibitors include omeprazole and esomeprazole. Treatment of the subject can also include administering an effective amount of a DPP-4 inhibitor. Examples of DPP-4 inhibitors include sitagliptin.

In embodiments, the subject in need is a Type 1 diabetic. In embodiments, the subject in need is a Type 2 diabetic. In embodiments, the subject in need also is a subject receiving a kidney transplant. In embodiments, the donor for the islet cells is also the donor for the kidney transplant.

### P Embodiments

Embodiment P1. A method of culturing islet cells, the method comprising: (a) obtaining islet cells from a donor; and (b) incubating the islet cells in a cell culture medium comprising a hydrogel and a gastrin compound.

Embodiment P2. The method of embodiment P1, wherein the gastrin compound is gastrin-34, gastrin-17, or gastrin-14.

Embodiment P3. The method of embodiment P2, wherein the gastrin compound is gastrin-34.

Embodiment P4. The method of embodiment P3, wherein the gastrin compound comprises the sequence of SEQ ID NO:1 or SEQ ID NO:2.

Embodiment P5. The method of embodiment P2, wherein the gastrin compound is gastrin-17.

Embodiment P6. The method of embodiment P5, wherein the gastrin compound comprises the sequence of SEQ ID NO:3 or SEQ ID NO:4.

Embodiment P7. The method of any of embodiments P1 to P6, wherein the concentration of gastrin is about 100 nM to about 600 nM.

Embodiment P8. The method of any of embodiments P1 to P7, wherein the hydrogel comprises a soft polysaccharide hydrogel capable of conversion to a hard polysaccharide hydrogel, the soft polysaccharide hydrogel comprising: (a) one or more water soluble high acyl gellan gum polymers; (b) one or more water soluble low acyl gellan gum polymers; and (c) one or more water soluble chemically modified gellan gum polymers or one or more peptide modified gellan gum polymers, wherein the soft polysaccharide hydrogel exhibits a homogeneous matrix structure and the hard polysaccharide hydrogel exhibits an aggregated matrix network structure.

Embodiment P9. The method of any of embodiments P1 to P8, wherein the incubating is for more than three days.

Embodiment P10. The method of embodiment P9, wherein the incubating is for at least a week.

Embodiment P11. The method of embodiment P10, wherein the incubating is for at least two weeks.

Embodiment P12. The method of embodiment P10, wherein the incubating is for at least three weeks.

Embodiment P13. The method of embodiment P10, wherein the incubating is for at least four weeks.

Embodiment P14. The method of embodiment P10, wherein the incubating is for about two weeks to about four weeks.

Embodiment P15. A method of treating diabetes in a subject in need thereof, the method comprising: (a) culturing islet cells according to any of the methods of embodiments P1 to P14, thereby obtaining cultured islet cells; and (b) administering to the subject an effective amount of the cultured islet cells.

Embodiment P16. The method of embodiment P15, further comprising administering to the subject an effective amount of a gastrin compound.

Embodiment P17. The method of embodiment P16, wherein the gastrin compound is administered post administration of the islet cell transplant.

Embodiment P18. The method of embodiment P16, wherein the gastrin compound is administered pre administration of the islet cell transplant.

Embodiment P19. The method of embodiment P16, wherein the gastrin compound is administered to the subject daily for about 30 days.

Embodiment P20. The method of embodiment P16, wherein the gastrin compound is administered to the subject twice daily for about 30 days.

Embodiment P21. The method of any of embodiments P16 to P20, wherein the gastrin compound is gastrin-34, gastrin-17, or gastrin-14

Embodiment P22. The method of any of embodiments P16 to P20, wherein the gastrin compound is gastrin-17.

Embodiment P23. The method of any of embodiments P16 to P22, comprising administering to the subject about 0.01 microgram to about 100 micrograms of the gastrin compound.

Embodiment P24. The method of any of embodiments P15 to P23, wherein the diabetes is Type 1 diabetes.

Embodiment P25. The method of any of embodiments P15 to P23, wherein the diabetes is a Type 2 diabetes.

Embodiment P26. An islet cell culture comprising islet cells, a hydrogel, a gastrin compound, and a culture medium.

Embodiment P27. An islet cell obtained by the method of any of embodiments P1-P14.

Embodiment P28. A kit for culturing islet cells comprising a hydrogel and a gastrin compound.

### Embodiments

Embodiment 1. A method of culturing islet cells, the method comprising: (a) obtaining islet cells from a donor; and (b) incubating the islet cells in a cell culture medium comprising a hydrogel and a gastrin compound.

Embodiment 2. The method of embodiment 1, wherein the gastrin compound is gastrin-34, gastrin-17, or gastrin-14.

Embodiment 3. The method of embodiment 2, wherein the gastrin compound is gastrin-34.

Embodiment 4. The method of embodiment 3, wherein the gastrin compound comprises the sequence of SEQ ID NO:1 or SEQ ID NO:2.

Embodiment 5. The method of embodiment 2, wherein the gastrin compound is gastrin-17.

Embodiment 6. The method of embodiment 5, wherein the gastrin compound comprises the sequence of SEQ ID NO:3 or SEQ ID NO:4.

Embodiment 7. The method of any of embodiments 1 to 6, wherein the concentration of gastrin is about 100 nM to about 600 nM.

Embodiment 8. The method of any one of embodiments 1 to 7, wherein the cell culture medium comprises trehalose.

Embodiment 9. The method of embodiment 8, wherein the concentration of trehalose is between about 0.01 M to about 0.35 M.

Embodiment 10. The method of any of embodiments ito 9, wherein the hydrogel comprises a soft polysaccharide hydrogel capable of conversion to a hard polysaccharide hydrogel, the soft polysaccharide hydrogel comprising: (a) one or more water soluble high acyl gellan gum polymers; (b) one or more water soluble low acyl gellan gum polymers; and (c) one or more water soluble chemically modified gellan gum polymers or one or more peptide modified gellan gum polymers, wherein the soft polysaccharide hydrogel exhibits a homogeneous matrix structure and the hard polysaccharide hydrogel exhibits an aggregated matrix network structure.

Embodiment 11. The method of any of embodiments ito 10, wherein the incubating is for more than three days.

Embodiment 12. The method of embodiment 11, wherein the incubating is for at least a week.

Embodiment 13. The method of embodiment 12, wherein the incubating is for at least two weeks.

Embodiment 14. The method of embodiment 12, wherein the incubating is for at least three weeks.

Embodiment 15. The method of embodiment 12, wherein the incubating is for at least four weeks.

Embodiment 16. The method of embodiment 12, wherein the incubating is for about two weeks to about four weeks.

Embodiment 17. A method of treating diabetes in a subject in need thereof, the method comprising: (a) culturing islet cells according to any of the methods of embodiments 1 to 16, thereby obtaining cultured islet cells; and (b) administering to the subject an effective amount of the cultured islet cells.

Embodiment 18. The method of embodiment 17, further comprising administering to the subject an effective amount of a gastrin compound.

Embodiment 19. The method of embodiment 18, wherein the gastrin compound is administered post administration of the islet cell transplant.

Embodiment 20. The method of embodiment 18, wherein the gastrin compound is administered pre administration of the islet cell transplant.

Embodiment 21. The method of embodiment 18, wherein the gastrin compound is administered to the subject daily for about 30 days.

Embodiment 22. The method of embodiment 18, wherein the gastrin compound is administered to the subject twice daily for about 30 days.

Embodiment 23. The method of any of embodiments 18 to 22, wherein the gastrin compound is gastrin-34, gastrin-17, or gastrin-14

Embodiment 24. The method of any of embodiments 18 to 22, wherein the gastrin compound is gastrin-17.

Embodiment 25. The method of any of embodiments 18 to 22, comprising administering to the subject about 0.01 microgram to about 100 micrograms of the gastrin compound.

Embodiment 26. The method of any of embodiments 17 to 25, wherein the diabetes is Type 1 diabetes.

Embodiment 27. The method of any of embodiments 17 to 25, wherein the diabetes is Type 2 diabetes.

Embodiment 28. An islet cell culture comprising islet cells, a hydrogel, a gastrin compound, and a culture medium.

Embodiment 29. An islet cell obtained by the method of any of embodiments 1-16.

Embodiment 30. A kit for culturing islet cells comprising a hydrogel and a gastrin compound.

Embodiment 31. The kit of claim 30, further comprising trehalose.

### EXAMPLES

### Example 1: 3D-Hydrogel Culture Improved Islet Mass Recovery During 4-Weeks Culture

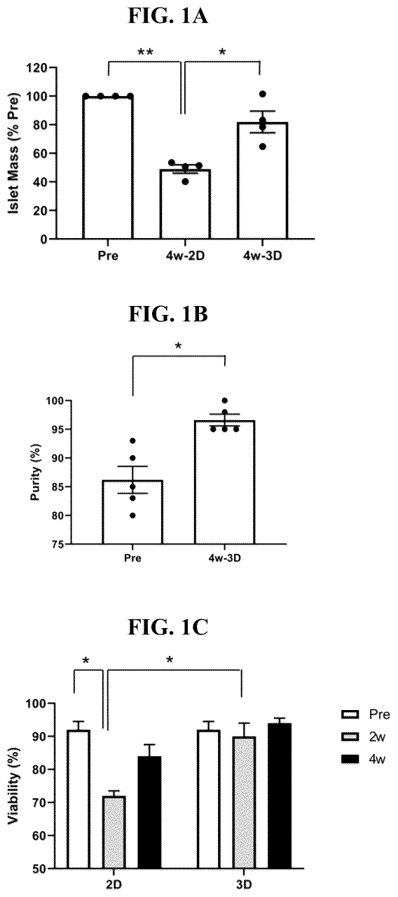

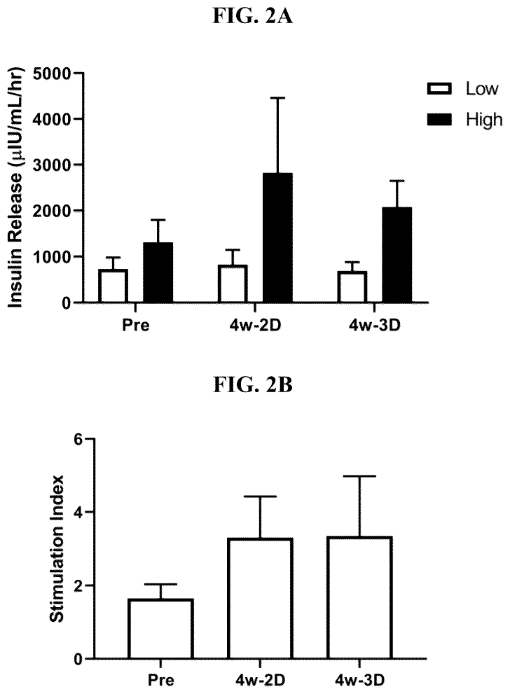

The 3D-islet culture method provided maintains islet morphological structure with >80% cell mass recovery (FIG. 1A) with increased islet purity as compared to pre-culture (FIG. 1), and maintains >90% viability (FIG. 1C) after 4-weeks culture (4w-3D) under physiological temperature and oxygen conditions. In contrast, culturing islet cells for 4 weeks in conventional 2D-culture (4w-2D) form clumps, significantly reduces the cell mass recovery (FIG. 1A), and decreases islet viability (FIG. 1C) as compared to pre-culture (FIGS. 1A and 1C). The function of islets cultured for four weeks was assessed by the glucose-stimulated insulin release assay. Both 4w-2D and 4w-3D culturing methods maintained islet function in vitro with no differences in the stimulation index between the groups (FIG. 2A-B).

After 4-weeks in 3D-islet culture, expression of proinflammatory cytokine genes ILIB (P<0.01, n=5, FIG. 3A), TNF (P<0.01, n=5, FIG. 3B), IL6 (P<0.05, n=5, FIG. 3C), and IL8 (P<0.05, n=5, FIG. 3D) and proapoptotic gene BBC3(P<0.05, n=5, FIG. 3E) is significantly decreased and islet function is maintained in vitro as compared to pre-culture (n=5).

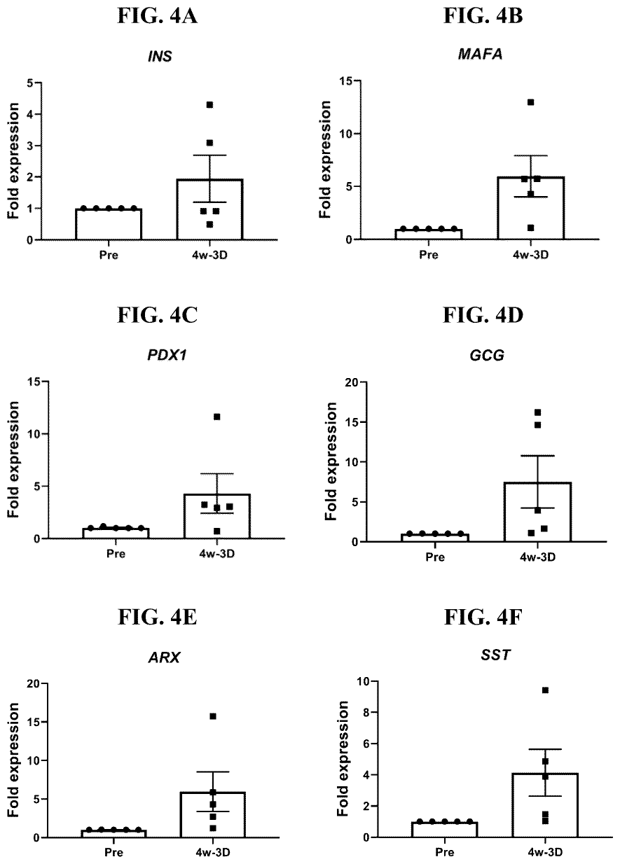

Culturing islets in 3D-hydrogel for 4-weeks did not alter the expression of R cell-associated genes INS (n=5, FIG. 4A), MAFA (n=5, FIG. 4B), and PDX1 (n=5, FIG. 4C). In addition 4-week culturing in 3D-hydrogel did not alter a cell-associated genes GCG (n=5, FIG. 4D) and ARX (n=5, FIG. 4E). Finally, 4-week 3D-hydrogel culture did not alter 6 cell-associated gene SST (n=5, FIG. 4F). This disclosed method also maintains β-cell mass and provides similar functionality to short term (3-day) culture. This disclosed culture method maintains islet cell viability and function in vitro for up to at least 8-weeks with slightly reduced cell mass recovery.

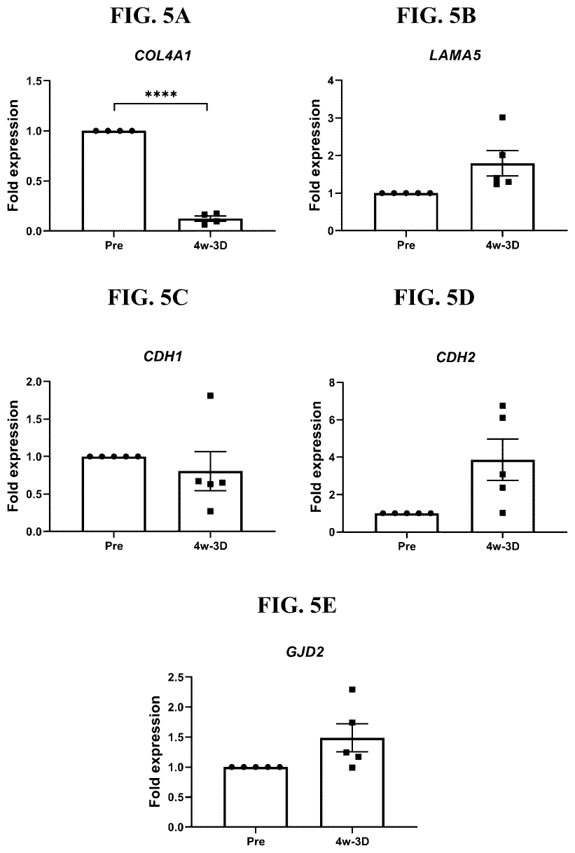

4-week culturing of islet cells in a 3D-hydrogel decreases the expression of extracellular matrix genes COL4A1 (FIG. 5A). The expression of other extracellular matrix genes LAMA5 (FIG. 5B), and CDH1 (FIG. 5C), CDH2 (FIG. 5D), and GJD2 (FIG. 5E) was not affected.

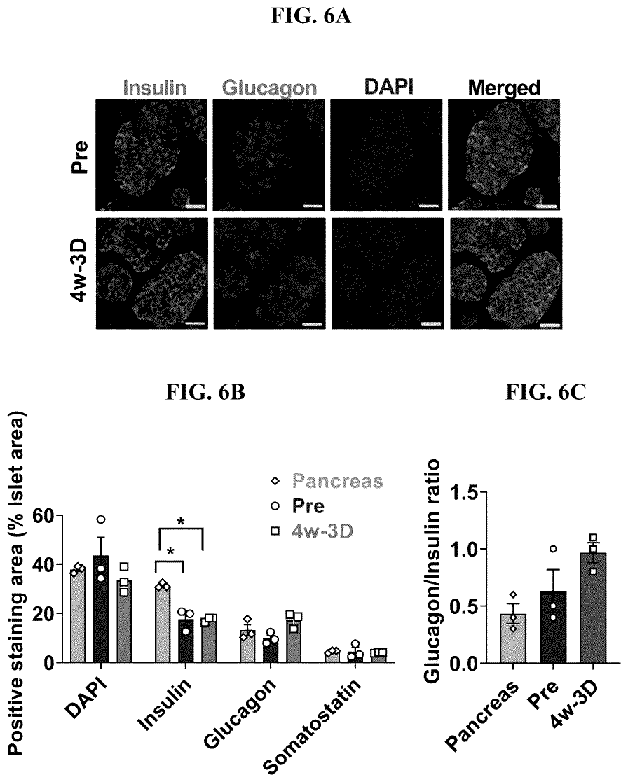

Immunohistochemistry was used to evaluate the endocrine cell composition and quantify the number of cell types in islets cultured for 4 weeks in 3D-hydrogel (FIG. 6A). The β cells mass, as indicated by the relative area of insulin staining per islet, was decreased in 4w-3D islets compared to the native pancreas (FIG. 6B). However, there was increase in a cells mass (relative area of glucagon staining per islet) in 4w-3D cultures (FIG. 6B-C). There was no change in the relative area stained with somatostatin, indicating no change in δ cell mass (FIG. 6B).

### Example 2: Gastrin Treatment Further Improved Islet Mass Recovery in 4-Week 3D-Hydrogel Culture

The addition of gastrin, a gastric hormone responsible for the secretion of gastric acid, in the islet culture media prevents decrease of islet mass recovery during the 4-weeks 3D-culture.

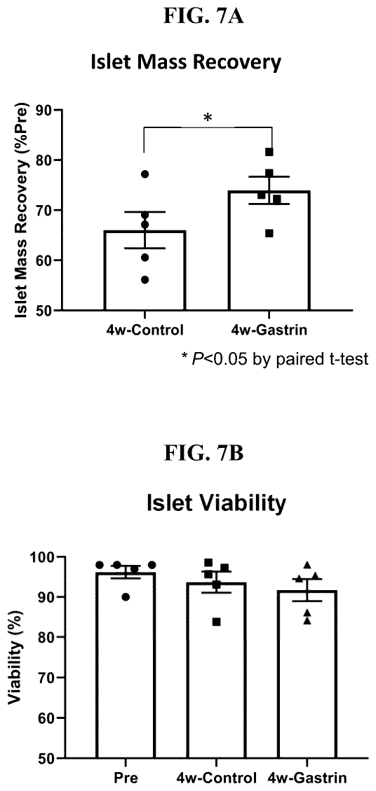

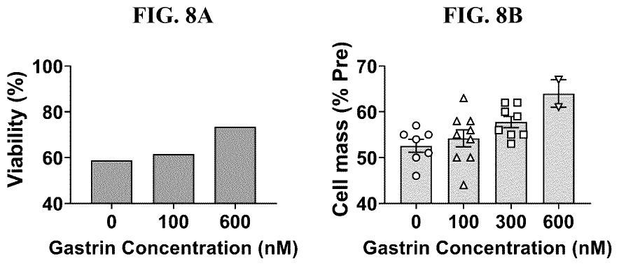

The morphology, cell mass recovery and viability of 4-weeks cultured islets in 3D hydrogel with 100 nM gastrin (4w-Gastrin) and without gastrin (4w-Control) were compared with those in islets before the long-term culture (Pre). The morphology of islets was well maintained without clumping and shown to have improved islet purity in both 4w-Control and 4w-Gastrin groups. The islet mass recovery was significantly higher in 4w-Gastrin group than 4w-Control group (73±6% vs. 66±8% respectively, n=5, P<0.05), indicating improved islet survival by the addition of gastrin in the culture media (FIG. 7A). There was no significant change in islet viability before culture, after 4-weeks culture without gastrin or with gastrin (96±4%, 94±6%, and 92±6% respectively) (FIG. 7B). Due to the improved mass recovery in 3D-hydrogel, islets in 2D culture were treated with different concentrations of gastrin (0-600 nN) for 3 weeks. The addition of gastrin improved islet viability (FIG. 8A) and mass recovery (FIG. 8B) in 2D culture.

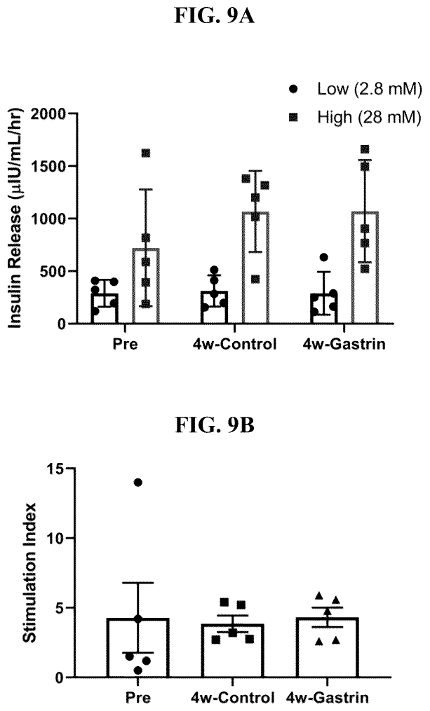

The function of 4-week cultured islets maintained in the presence (4w-Gastrin) or absence (4w-Control) of gastrin was compared with pre-culture islets using the glucose-stimulated insulin release assay. The islets showed a similar amount of insulin release with no difference in stimulation index among the groups (FIGS. 9A and 9B).

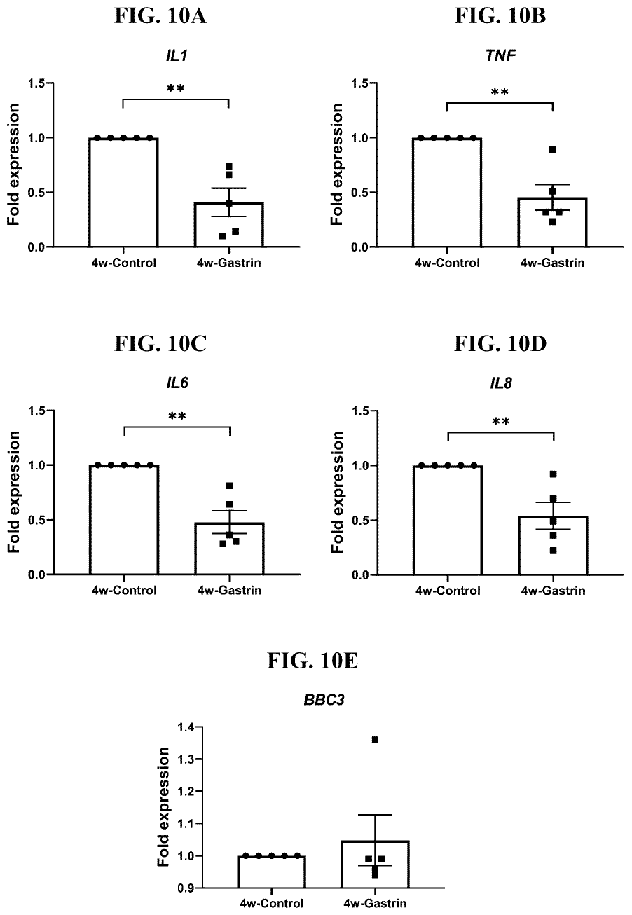

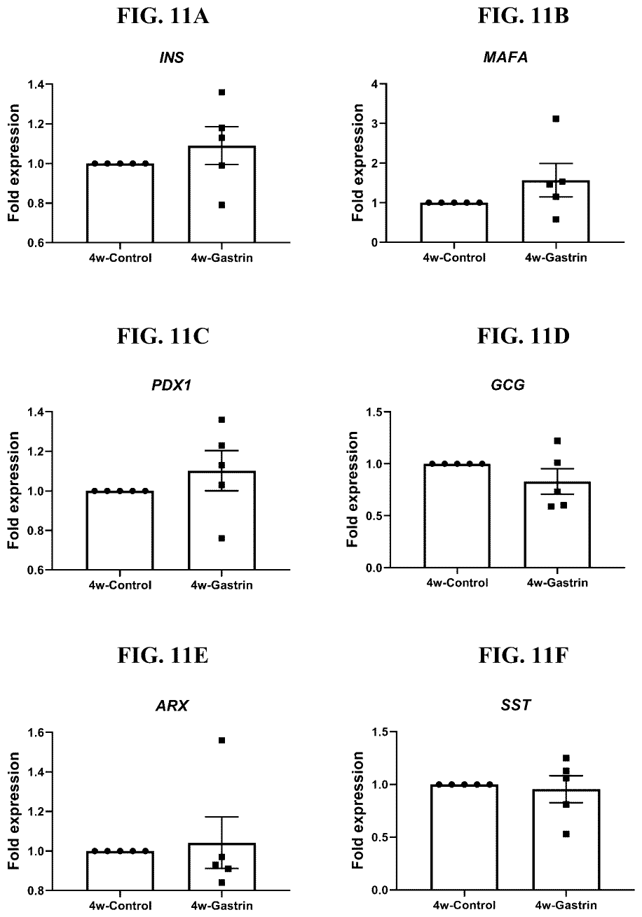

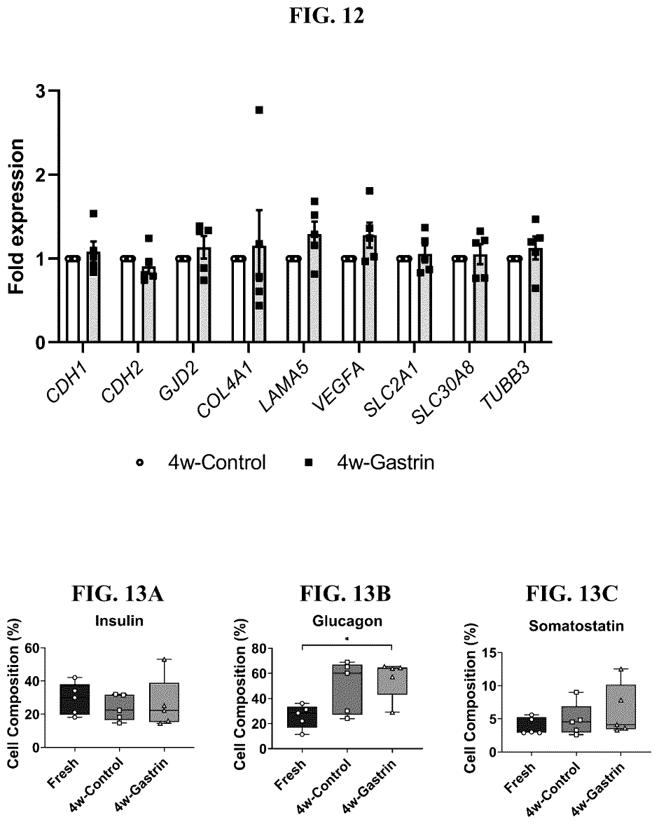

The gene expression of proinflammatory and proapoptotic genes in 4-week cultured islets in 3D hydrogel in the presence of gastrin (4w-Gastrin) was compared to cultured islets in the absence of gastrin (4w-Control). The addition of gastrin further reduces expression of proinflammatory cytokine genes IL1B (P<0.01, n=5, FIG. 10A), TNF (P<0.01, n=5, FIG. 10B), IL6 (P<0.01, n=5, FIG. 10C), and IL8 (P<0.01, n=5, FIG. 10D), but does not affect proapoptotic gene BBC3 expression (n=5, FIG. 10E). Addition of gastrin did not affect the gene expression of endocrine cell-associated genes: INS (n=5, FIG. 11A), MAFA (n=5, FIG. 11B), PDX1 (n=5, FIG. 11C), GCG (n=5, FIG. 11D), ARX (n=5, FIG. 11E), and SST (n=5, FIG. 11F). Moreover, gastrin treatment did not affect expression of extracellular matrix genes (FIG. 12).

The Milo-scWestern blot assay was used to evaluate islet composition after being cultured 4 weeks in 3D-hydrogel either in the presence or absence of gastrin (FIG. 13). The percentage of cells expressing glucagon (αcell marker), insulin (β cell marker), and somatostatin (6 cell marker) were similar between the islets before and after 4 weeks of culture in a 3D-hydrogel, however, the glucagon-expressing cells had a wider range (FIG. 13B). With the addition of gastrin to the culture media, the percentage of glucagon-expressing cells was significantly more than in fresh preparations (FIG. 13B).

### Example 3: Islets Cultured in 3D-Hydrogel for 4 Weeks Restored Normoglycemia in Diabetic Mice

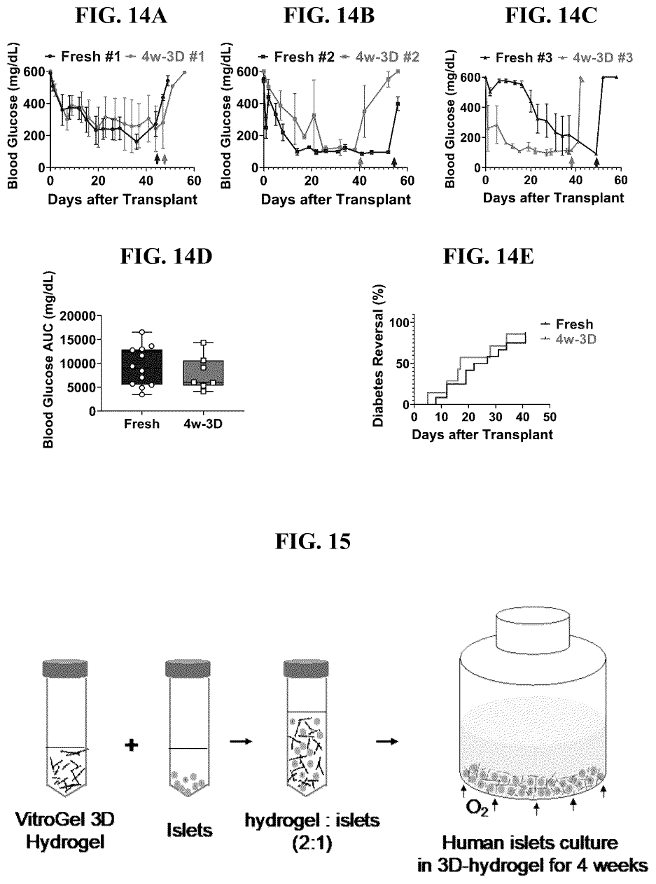

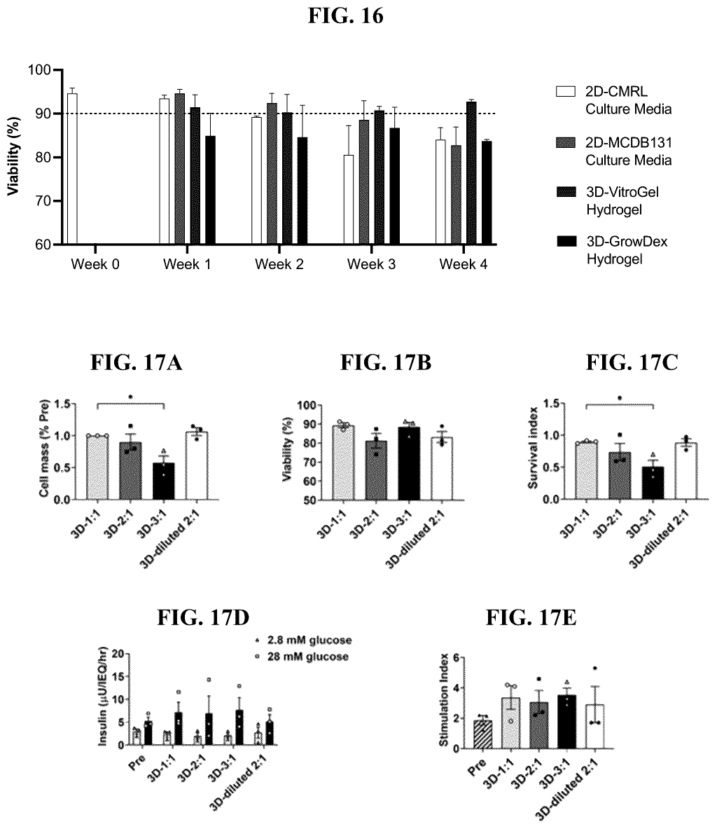

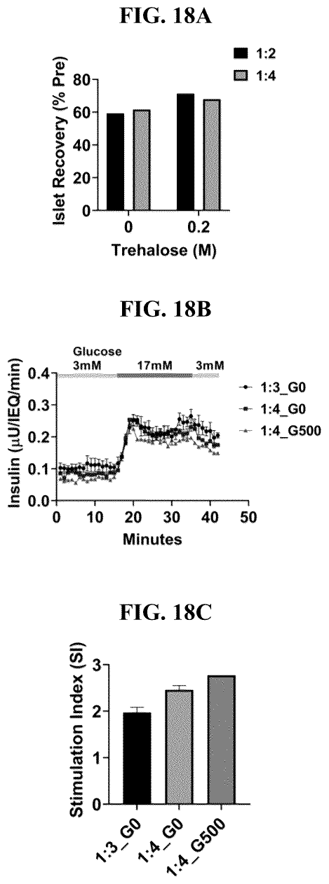

Human islets (1200 IEQ) cultured in 3D-hydrogel for 4 weeks were transplanted into the subcapsular area of the kidney of diabetic, nonobese combined immunodeficient diabetic (NOD SCID) mice (FIG. 14). 4w-3D islets restored normoglycemia in 71.4% ( 5/7) of transplanted NOD SCID mice, which recapitulated the restoration of normoglycemia in 66.7% of NOD SCID mice using freshly isolated islets (FIG. 14E). In addition, there was no significant difference in the area under the curve (AUC) of blood glucose levels between 4w-3D cultured and fresh islets one month after transplantation (FIG. 14D). This restoration of normoglycemia could be reversed by removing the grafted islet via nephrectomy (arrows in FIG. 14A-C), indicating the diabetes reversal was graft dependent (FIG. 14A-C).

### Example 4: Materials and Methods

Human Islets

Human islets were isolated by Southern California Islet Cell Resources Center (SC-ICRC) at the City of Hope, and distributed through the SC-ICRC islet distribution program, one to three days after islet isolation. The non-diabetic donors with hemoglobin Alc between 5.0 to 9.6 and age between 28 to 59 were used for this study. The demography of the islet donors used for this study was summarized in Table 1.

TABLE 1
Demography of islet donors

| | | | | | | Warm | Cold |
|---|---|---|---|---|---|---|---|
| | | | | HbA1c | Cause of | ischemia | Ischemia |
| Donor # | Age | Sex | BMI | (%) | death | (min) | (hour) |
| #1 | 28 | M | 21.0 | 5.5 | Anoxia | 0 | 8.5 |
| #2 | 34 | M | 28.1 | 5.6 | Head Trauma | 10 | 8.9 |
| #3 | 50 | F | 39.1 | 5.0 | CVA/Stroke | 0 | 11.6 |
| #4 | 49 | M | 34.8 | 5.5 | CVA/Stroke | 0 | 6.3 |
| #5 | 31 | M | 27.0 | 5.2 | Head Trauma | 0 | 11.4 |
| #6 | 30 | F | 30.7 | 5.1 | Head Trauma | unknown | 8.6 |
| #7 | 45 | F | 26.3 | 5.7 | CVA/Stroke | 8 | 5.4 |
| #8 | 46 | M | 33.2 | 5.5 | Head Trauma | 22 | 4.6 |
| #9 | 51 | F | 43.3 | 9.6 | Anoxia | 29 | 5.6 |
| #10 | 46 | M | 34.4 | 5.6 | Head Trauma | 0 | 9.8 |
| #11 | 33 | M | 30.3 | 4.9 | CVA/Stroke | 0 | 8.6 |
| #12 | 43 | M | 34.9 | 6.0 | CVA/Stroke | 0 | 8.2 |
| #13 | 44 | M | 20.3 | 5.2 | Head Trauma | 0 | 6.6 |
| #14 | 53 | M | 25.1 | 4.9 | CVA/Stroke | DCD | 5.6 |
| #15 | 44 | F | 23.0 | 6.2 | CVA/Stroke | 15 | 6.9 |
| #16 | 52 | M | 27.2 | 5.7 | CVA/Stroke | 0 | 7.1 |
| #17 | 37 | M | 24.2 | 5.5 | Head Trauma | 0 | 13 |
| #18 | 51 | M | 26.7 | 5.7 | Anoxia | DCD | 6.4 |
| #19 | 44 | F | 38.7 | 5.9 | CVA/Stroke | 0 | 7.0 |
| #20 | 45 | M | 32.8 | 5.4 | CVA/Stroke | 0 | 6.4 |
| #21 | 59 | F | 34.3 | 5.3 | CVA/Stroke | 0 | 9.5 |
| #22 | 28 | M | 30.7 | 5.2 | Head Trauma | DCD | 8.4 |
| #23 | 34 | M | 28.2 | 5.6 | Head Trauma | 10 | 8.9 |
| #24 | 50 | F | 39.1 | 5.0 | CVA/Stroke | 0 | 11.6 |

CVA: Cardiovascular accident, DCD: Donation after circulatory death

2D Islet Culture

Human islets 2-4 days after isolation (approximately 150-200 IEQ) were placed in standard 24-well suspension culture plate (Fisher Scientific, Hampton, NH) and 0.5 mL of serum-free CMRL 1066 supplemented CIT Modification media (CORNING, Corning, NY, USA) containing 0.1 μg/mL IGF-1 (Cell Sciences, Newburyport, MA, USA), 10 U/mL heparin Sodium (SAGENT Pharmaceuticals, Schaumburg, IL) and 0.5% human serum albumin was added to each well. After plating, the islets were placed in a 37° C. incubator with 21% O₂ air with 5% CO₂ for four weeks. Culture medium was changed every three to four days.

Islet Culture in 3D Hydrogel

The hydrogel for islet culture was prepared using commercially available polysaccharide hydrogel, VitroGel 3D (TheWell Bioscience, North Brunswick Township, NJ, USA), by diluting with sterile water with dilution ratio of 1:2 (v/v). Subsequently, the diluted hydrogel was mixed with equal volume of culture media containing islets (approximately 150-250 IEQ/well) and placed on a Millicell Cell Culture Insert with a pore size of 12 μm in a 24-well plate (PIXP01250, Millipore Sigma, Burlington, MA, USA). After ten minutes, 0.5 mL of serum-free CMRL 1066 supplemented CIT Modification media (CORNING, Corning, NY, USA) containing 0.1 μg/mL IGF-1 (Cell Sciences, Newburyport, MA, USA), 10 U/mL heparin Sodium (SAGENT Pharmaceuticals, Schaumburg, IL) and 0.5% human serum albumin with or without 100 nmol/L [Leu15]-gastrin I (Sigma, St. Louis, MO, USA) was added to each well and cultured in a 37° C. incubator containing the air with 5% CO2 for four weeks. Culture medium was changed every three to four days. At the end of islet culture, islets were collected from the hydrogel by the gentle pipetting using a 1.0 mL pipette tip into a 1.5 mL microcentrifuge tube.

Gene Expression Measured by RT-PCR

In order to determine the effects of culturing islet cells in 3D-hydrogel on gene expression, RNA was extracted from pre-culture and 4-week 3D-hydrogel cultured islets with or without gastrin using TRI REAGENT (Molecular Research Center, Inc., Cincinnati, OH). A total of 1.0 μg RNA from each sample was reverse-transcribed into cDNA using the Maxima First Strand cDNA Synthesis Kit (Thermo Fisher Scientific, Waltham, MA). The final cDNA products were diluted by adding 60 μL of H₂O. Relative gene expression of target mRNA in pre- and 4-week 3D-hydrogel cultured islets (n=5 per group) was assessed with RT-PCR using the TaqMan® Gene Expression Assay as listed in Table 2 (Applied Biosystems, Foster City, CA). Assays were performed in 10 μL duplicate reactions containing TaqMan® Fast Advanced Master Mix, 20× TaqMan® Gene Expression Assay Mix, and 1 μL cDNA. All PCR reactions were performed in a Real Time PCR 7500 system and results were analyzed with the Sequence Detection Software version 2.3 of the system (Applied Biosystems, Foster City, CA) using the 2^{-ΔΔCT} method. Human β-actin was used as an endogenous control gene to correct for variations in input RNA and cDNA amplification of different samples.

TABLE 2
TaqMan ® Gene Expression Assay

| Gene Symbol | Assay ID/Item# |
|---|---|
| ACTB | 4326315E |
| IL1B | Hs00174097_m1 |
| TNF | Hs00174128_m1 |
| IL6 | Hs99999032_m1 |
| IL8 | Hs00174103_m1 |
| BBC3 | Hs00248075_m1 |
| INS | Hs02741908_m1 |
| MAFA | Hs01651425_s1 |
| PDX1 | Hs00236830_m1 |
| GCG | Hs01031536 |
| ARX | Hs00292465_m1 |
| SST | Hs00356144_m1 |
| COL4A1 | Hs00266237_m1 |
| LAMA5 | Hs00966585_m1 |
| CDH1 | Hs01023895_m1 |
| CDH2 | Hs00983056_m1 |
| GJD2 | Hs00950432_m1 |
| VEGFA | Hs00900055_m1 |
| SLC2A1 | Hs00892681_m1 |
| SLC30A8 | Hs00545183_m1 |
| TUBB3 | Hs00801390_s1 |

Measurement of Islet Mass Recovery

Recovered islet cells were photomicrographed before and after 4-weeks of culture using an Infinity 2 camera with Olympus stereomicroscope, Olympus SZ (Olympus America, Center Valley, PA, USA). The area of islet cells in each photomicrograph was measured using Olympus CellSens software (Olympus America), and the islet mass recovery was calculated by the total islet area after four-weeks culture divided by total islet area of pre-culture in the corresponding sample.

Islet Viability and Morphology Assessment

Islet cells were stained with 10 μg/mL propidium iodide (PI) (Millipore Sigma, St. Louis, MO, USA) and 10 μg/mL of Hoechst 33342 (HO) (Thermo Fisher Scientific, Waltham, MA) for 5 minutes at room temperature, washed with PBS, then placed on a 6-well plate. The images of PI/HO staining were taken under a IXDP50 Olympus fluorescent microscope with DP74 camera using a 4× objective lens (Olympus America). Viability was determined by the area of PI staining divided by the area of HO staining analyzed by CellSens software. For the experiment with gastrin culture, 0.5 μM Fluorescein diacetate (FDA) solution (Millipore Sigma) was used instead of HO to determine the viable total islets. The viability was determined by the area of PI staining divided by the total islet area analyzed by CellSens software. Islet morphology was evaluated by dithizone (DTZ) staining.

In Vitro Islet Function by Glucose-Stimulated Insulin Secretion (GSIS) in a Static Incubation

Islet cells were pre-incubated with RPMI 1640 media containing 10 mM HEPES, 10% Fetal bovine serum (FBS), and 3 mM glucose for 4 hours prior to assay. Islet cells (120 IEQ/sample) were incubated in a Millicell Cell Culture Insert, 12 mm, polycarbonate, a pore size of 12 μm (PIXP01250, Millipore Sigma) placed in a 24-well plate with 0.5 mL of 2.8 mM Krebs Ringer Buffer (KRB) solution for 30 minutes followed by 28 mM KRB solution for 30 minutes. Samples were collected at the end of each incubation, and insulin contents of the samples were measured by the insulin ELISA kit specific to human insulin (Mercodia, Winston Salem, NC, USA). Stimulation index was calculated by the insulin release during the incubation in the 28 mM KRB solution divided by the insulin release in 2.8 mM KRB solution of the corresponding sample.

Immunohistochemistry

Islets and pancreatic tissue were fixed with 10% formalin, embedded in 3% agarose Type VII (Sigma) (in case of islets), and processed for paraffin embedding. The sections of paraffin-embedded islets or pancreatic tissue were immunostained for insulin (guinea pig anti-insulin; Dako, Santa Clara, CA), glucagon (mouse anti-glucagon; Sigma), somatostatin (rabbit anti-somatostatin; Dako, Santa Clara, CA) using the secondary antibodies conjugated to Alexa fluorophores (Jackson ImmunoResearch, West Grove, PA) and counterstained with DAPI (Santa Cruz Biotechnology, Dallas, TX) for DNA. Images were captured using a 20× objective and Zeiss LSM 700 Confocal Microscope (Zeiss, Whiteplains, NY), and the percentage of endocrine hormone-positive cells in an islet was determined by the positive staining area divided by the total area of the corresponding islet on the merged staining image. The average of a total 20-50 islets/group/batch was analyzed. Any cell cluster having no insulin, glucagon, or somatostatin positive cells was considered a non-islet and not included in the analysis.

Milo-scWestern Blot

Islets were dispersed in a single cell suspension by incubating with 1 mL of 0.025% trypsin in PBS at 37° C. water bath for 10 minutes. 1 mL of cell suspension (1.0×10⁵ cells/mL), either from fresh islet isolation or from islets cultured for 4 weeks in 3D-hydrogel, was placed on an scWest chip where individual cells settle into microwells. The scWest chip is then placed in a Milo single-cell Western system (Protein Simple, San Jose, CA). The Milo lyses the cells, runs an SDS-PAGE separation on each of the single-cell lysate, and immobilizes the proteins. Then the chips were probed for 1-2 hours with the following primary antibodies diluted in antibody dilution buffer (ProteinSimple); guinea pig anti-insulin (1:100, Dako A0564, Santa Clara, CA, USA), rabbit anti-somatostatin (1:50, Dako A0566), mouse anti-glucagon (1:10, BD biosciences 565859, Franklin Lakes, NJ, USA), rabbit anti-beta-actin (1:20, Cell Signaling 49675S, St. Louis, MO, USA). The chips were then washed three times with wash buffer (ProteinSimple) and incubated with secondary antibodies conjugated to Alexa fluorophores or Cy3 (1:25, Jackson ImmunoResarch, West Grove, PA). Subsequently, chips were washed, then stained with 1 mg/mL DAPI (Santa Cruz Biotechnology, Dallas Tx, U.S.A.) for 5 minutes. The scWest chip was placed in Innoscan 710 microarray scanner (Innopsys, Carbonne, France) to measure chip fluorescence and results were analyzed with Scout Software (Protein Simple, San Jose, CA).

Human Islet Transplantation in NOD SCID Mice

10-12 week old male NOD SCID mice were treated with 50 mg/kg streptozotocin via a daily intraperitoneal injection for three consecutive days. Mice that were hyperglycemic (>350 mg/dL) for two consecutive days were considered diabetic and used as recipients for islet transplantation. Fresh human islets and islets cultured for 4 weeks in 3D-hydrogel (1200 IEQ) were transplanted under the left kidney capsule of diabetic mice. Blood glucose was sampled 2-3 times per week. Diabetes was considered reversed in recipient mice that maintained a blood glucose less than 200 mg/dL for more than two consecutive measurements. At the end of each experiment, a nephrectomy was performed to remove the implanted islets and confirm graft dependence of diabetes reversal. The area under the curve (AUC) as a sum of the blood glucose between day 0 to 28 was calculated to assess the overall islet function in vivo.

### Example 5: GAST-17 Treatment Improves Clinical Islet Transplantation

In a clinical trial with Type I diabetic patients, GAST-17, an FDA-approved gastrin analogue, was used in a combination treatment with islet transplantation. GAST-17 was administered at the time of islet implantation and after. 75% (¾) of the patients completed the GAST-17 treatment and those 3 patients attained insulin freedom with an average of 390,000 IEQ (Table 3). This is an improvement compared to a non-gastrin clinical trial in which about 850,000 IEQ was needed to achieve a similar result.

TABLE 3
Depicts clinical trial data using GAST-17 treatment
in clinical islet transplantation.

| | |
|---|---|
| # IT recipients | 4 |
| Insulin free subjects | 3/4 |
| Islet infusions to insulin free | 1 |
| Total IEQ to insulin free | ~390,000 |
| Total IEQ/kg body weight | 4,872 |
| Independence for 1 year | 3 |

### Example 6: Materials and Methods

Long-Term Human Islet Culture

COH (City of Hope) procedure is described herein for culturing human pancreatic islets for clinical or research use during the manufacture of islet products.

Reagents and Materials

TABLE 4
Reagents

| | MANUFACTURER/ | |
|---|---|---|
| ITEM | DISTRIBUTOR | PART # |
| CMRL 1066, Supplemented | SC-ICRC | N/A |
| CIT Modification | | [SOP-1119] |
| Gastrin | SC-ICRC | N/A |
| Water for injection (WFI)**, | COH Pharmacy* | NDC- |
| sterile | | 040904887-10* |
| VitroGel ® 3D hydrogel | TheWell Bioscience | TWG001 |
| VitroGel ® MMP hydrogel | TheWell Bioscience | TWG010 |
| VitroGel ® Dilution Solution | TheWell Bioscience | MS02-100 |
| VitroGel ® Cell Recovery | TheWell Bioscience | MS03-100 |
| Solution | | |
| IPA, 70% | EMD* | PX1840-4* |
| pH Calibrator, 7.0** | Fisher Scientific* | 13-641-275* |
| pH Calibrator, 10.0** | Fisher Scientific* | 13-641-276* |
| pH Calibrator, standard 7.38** | Fisher Scientific* | 1563-16* |
| Water for Irrigation, Sterile** | Baxter* | 2F7114* |
| 0.9% Sodium Chloride Solution | COH Pharmacy* | N/A |
| Injection | | |
| COHSII Media** | Gemini* | 900-750* |
| COHSII Modified Media** | Gemini* | 900-752* |
| COHITM Modified #1 | Gemini* | 900-753* |
| Human Serum Albumin-HSA, | COH Pharmacy* | NDC-0944- |
| 25% (100 mL flexpack) | | 0493-02* |

*Or Equivalent.
**Optional.

TABLE 5
Equipment

| ITEM | MANUFACTURER/DISTRIBUTOR* |
|---|---|
| BSC (Thermo 1300 Series A2 | Thermo Fisher Scientific |
| and Herasafe KS) | |
| Centrifuge - Beckman J6** | Beckman |
| CO₂ Incubator | Sanyo Scientific and Thermo |
| | Fisher Scientific |
| pH Meter** | Corning |
| Pipet-Aid (PipetBoy) | Integra Biosciences |
| Tube Sealer** | Sebra |

*Or Equivalent.
**Optional.

TABLE 6
Reusables

| ITEM | MANUFACTURER/DISTRIBUTOR* |
|---|---|
| Beaker (1-2 L)** | Fisher Scientific |
| Clamp, 3-prong** | VWR |
| Conical Tube Rack, 50 mL** | Fisher Scientific |
| Conical Tube Rack, 250 mL | Fisher Scientific |
| Marker | VWR |
| Ring Stand with Rod** | Fisher Scientific |

*Or Equivalent.
**Optional.

TABLE 7
Disposables

| | MANUFACTURER/ | |
|---|---|---|
| ITEM | DISTRIBUTOR* | PART #* |
| 6-well plate** | Corning | 3516 |
| Bag, Platelet Storage, 1000 mL** | Fenwal | 4R2350 |
| Bottles, Plastic, 1 L** | Fisher Scientific | 09-761-10 |
| Cable Ties** | McMaster Carr | 7130K12 |
| Conical Tube(s), 50 mL** | Sarstedt | 62.547.004 |
| Conical Tube(s), 250 mL** | Fisher Scientific | 05-538-53 |
| Couplers** | Terumo BCT | 90904 |
| Drape, Medium Convertors, sterile | Cardinal Health | 9355 |
| Pipets, Serological, | Fisher Scientific | 13-675- |
| Aspiration 5 mL** | | 10CC |
| Pipets, Serological, 1 mL*** | Fisher Scientific | 13-675-46 |
| Pipets, Serological, 2 mL* | VWR | 53300-363 |
| Pipets, Serological, 5 mL** | VWR | 53300-421 |
| Pipets, Serological, 10 mL | Fisher Scientific | 13-678-14A |
| wide mouth | | |
| Pipets Serological, 25 mL | VWR | 53106-195 |
| Syringe, 60 mL** | Fisher Scientific | 13-689-8 |
| Tissue Culture Flask, T-75** | Sarstedt | 83.1813.502 |
| Tissue Culture Flask, T-175 | Sarstedt | 83.1812.502 |
| G-Rex ® chamber | Wilsonwolf | P/N 80040S |
| | | P/N 80500 |

Day of Isolation (Day 0) Islets Mixed with Hydrogel and Culture Setup

Islet cell fractions were transferred to a G-Rex chamber for 3D culture and to T-175 flasks for 2D culture. Total islet cell equivalents (IEQs) were recorded for each fraction, including rescue fractions. VitroGel concentration was adjusted by diluting the VitroGel with VitroGel Dilution Solution or water for injection (sterile) (1:2 ratio; hydrogel: dilution solution or water). For 3D culture, islet cells were mixed with the diluted VitroGel to achieve no more than 100,000 islet cells in 450 mL per chamber, as shown in below table.

TABLE 8
Depicts mixtures of islet cells and diluted VitroGel 3D.

| | | | Total volume |
|---|---|---|---|
| Final | Maximum islets | Total number | of required |
| islet (IEQ) | culture in each | of chamber* | diluted |
| culture | chamber (IEQ) | use | hydrogel (mL) |
| <300,000 | 100,000 | 3 | 90 |
| 300,000-400,000 | 100,000 | 4 | 120 |
| 400,000-500,000 | 100,000 | 5 | 150 |
| >500,000 | 100,000 | 6 | 180 |

*Chamber volume capacity: 450 mL

The VitroGel was allowed to form a soft hydrogel at room temperature (approximately 10-30 min). Approximately 150-300 mL culture media was added to the top of the soft hydrogel. Chambers were then placed in an incubator.

Media Change

G-Rex chambers containing islets with hydrogel were transferred from incubator to a prepared Biological Safety Cabinet (BSC). Approximately 100-200 mL culture media was aspirated from each chamber. 5-10 mL removed culture media was aliquoted in multiple cryopreservation vials and stored at −80 degree Celsius. 100-200 mL freshly made culture media was added back to the chambers, which were then placed in an incubator. Media changes were performed twice per week.

Islet Harvest after Culture

For breaking down the hydrogel and harvesting the islets after culture, 0.9% NaCl based solution was developed and used. This solution (described as recovery solution) contained diluted 0.9% NaCl (up to 1:4 dilution with water for injection), HEPES (10-25 mM), human serum albumin (up to 4%), and Trehalose (up to 0.18 M). The recovery solution was warmed in a water bath before use. To harvest islets, culture media was removed first and 10 mL (G-rex 10) or 100 mL (G-rex 100) warmed recovery solution was added in the chambers. Keep in incubator for 5-10 minutes. Add additional 10 mL or 100 mL more the recovery solution in the chambers. Hydrogel and islet mixture was broken down using 10 mL pipet and transfer the mixture to a 50 mL conical tube (G-rex 10) or 250 mL (G-rex 100). The conical tubes containing islet cells and hydrogel mixture were centrifuged at 1000 rpm for 2 minutes. Supernatant was removed and re-centrifugated at 1000 rpm for 2 minutes. The islets pellets were combined and supplemented in the CMRL culture media in 50 mL conical tubes for later assays.

### Example 6: Comparison of Hydrogel Stiffness on Islet Survival and Function

FIGS. 17A-17C show the recovery index comparing the islet mass recovery (% pre culture) in different stiffness of Vitrogel 3D hydrogels after 4 weeks culture relative to the hydrogel condition that is diluted 1:2 ratio with sterile water then mixed equal volume of culture media containing islets (3D-1:1, condition presented as “4w-3D” throughout in this application) (FIG. 17A), viability (%) (FIG. 17B), and overall survival index calculated by the recovery index multiplied with viability (%) (FIG. 17C). Insulin release from islet cells in the glucose-stimulated insulin release assay (FIG. 17D) and the glucose stimulation index (FIG. 17E), comparing pre-culture islet cells to islets cultured 4 weeks in the different stiffness of Vitrogel 3D hydrogel. The hydrogel dilution and mixing ratio tested in this experiment are shown in Table 9 below.

TABLE 9
Example of different stiffness of Vitrogel
3D hydrogel for islet culture

| | 3D | 3D | 3D | 3D-diluted |
|---|---|---|---|---|
| Group | 1:1 | 2:1 | 3:1 | 2:1 |
| Gel dilution (stock hydrogel:water) | 1:2 | 1:2 | 1:2 | 1:3 |
| Mixing ratio (diluted | 1:1 | 2:1 | 3:1 | 2:1 |
| hydrogel:islets with media) | | | | |
| Stock hydrogel amount (μL) per | 25 | 33 | 37.5 | 25 |
| well (24-well insert) | | | | |

### Example 7: Optimizing 4w-3D Cultured Islets with Hydrogel Dilution and Gastrin

Method: Vitrogel 3D hydrogel (TheWell Bioscience) was pre-diluted with the water containing trehalose for the osmolarity adjustment at a different dilution ratio; 1:2, 1:3, or 1:4 (hydrogel: water, v/v). Then, the diluted hydrogel was mixed with the media containing the islets at a mixing ratio of 2:1 (hydrogel:media, v/v). The hydrogel-embedded islets were cultured with the CMRL islet culture media with or without 500 nmol/L[Leu15]-gastrin I (Sigma) for four weeks. Islet mass recovery was compared to pre-culture (% pre-culture) and function was assessed by the glucose-stimulated insulin release assay in a perifusion system.

Result: The islet recovery was higher when the trehalose was added to the water to dilute hydrogel (both 1:2 and 1:4) for osmolarity adjustment (A). The islet function was better in a 1:4 diluted hydrogel (1:4_G0) condition as compared to a 1:3 diluted hydrogel (1:3_G0) condition by lowered basal insulin release (B), resulting in a higher stimulation index (C). The addition of 500 nmol/L gastrin I (1:4_G500) further lowered basal insulin release, thus increasing the stimulation index.

What is claimed is:

1. A method of culturing islet cells, the method comprising:
(a) obtaining islet cells from a donor; and
(b) incubating the islet cells in αcell culture medium comprising a hydrogel and a gastrin compound.

2. The method of claim 1, wherein the gastrin compound is gastrin-34, gastrin-17, or gastrin-14.

3. The method of claim 2, wherein the gastrin compound comprises the sequence of SEQ ID NO:1, SEQ ID NO:2, SEQ ID NO:3, or SEQ ID NO:4.

4. The method of claim 1, wherein the concentration of gastrin is about 100 nM to about 600 nM.

5. The method of claim 1, wherein the cell culture medium further comprises trehalose.

6. The method of claim 5, wherein the concentration of trehalose is between about 0.01 M to about 0.35 M.

7. The method of claim 1, wherein the hydrogel comprises a soft polysaccharide hydrogel capable of conversion to a hard polysaccharide hydrogel, the soft polysaccharide hydrogel comprising:
(a) one or more water soluble high acyl gellan gum polymers;
(b) one or more water soluble low acyl gellan gum polymers; and
(c) one or more water soluble chemically modified gellan gum polymers or one or more peptide modified gellan gum polymers, wherein the soft polysaccharide hydrogel exhibits a homogeneous matrix structure and the hard polysaccharide hydrogel exhibits an aggregated matrix network structure.

8. The method of claim 1, wherein the incubating is for more than three days.

9. The method of claim 8, wherein the incubating is for at least a week, at least two weeks, at least three weeks, or at least four weeks.

10. The method of claim 9, wherein the incubating is for about two weeks to about four weeks.

11. A method of treating diabetes in a subject in need thereof, the method comprising:
(a) culturing islet cells according to claim 1, thereby obtaining cultured islet cells; and
(b) administering to the subject an effective amount of the cultured islet cells.

12. The method of claim 11, further comprising administering to the subject an effective amount of a gastrin compound.

13. The method of claim 12, wherein the gastrin compound is administered pre or post administration of the islet cell transplant.

14. The method of claim 12, wherein the gastrin compound is administered to the subject once or twice daily for about 30 days.

15. The method of claim 12, wherein the gastrin compound is gastrin-34, gastrin-17, or gastrin-14.

16. The method of claim 12, comprising administering to the subject about 0.01 microgram to about 100 micrograms of the gastrin compound.

17. The method of claim 11, wherein the diabetes is Type 1 diabetes or Type 2 diabetes.

18. An islet cell culture comprising islet cells, a hydrogel, a gastrin compound, and a culture medium.

19. An islet cell obtained by the method of claim 1.

20. A kit for culturing islet cells comprising a hydrogel and a gastrin compound.
